(19) United States
(12) Patent Application Publication
(10) Pub. No.: US 2006/0293377 A1
(43) Pub. Date: Dec. 28, 2006

(54) Amorphous and polymorphic forms of telmisartan sodium

(76) Inventors: Shlomit Wizel, Petah Tiqva (IL); Nurit Perlman, Kfar Saba (IL); Sharon Avhar-Maydan, Givataym (IL); Eyal Gilboa, Bat-Yam (IL)

Correspondence Address: KENYON & KENYON LLP, ONE BROADWAY, NEW YORK, NY, 10004, US

(21) Appl. No.: 11/267,520

(22) Filed: Nov. 3, 2005

Related U.S. Application Data
(60) Provisional application No. 60/624,842, filed on Nov. 3, 2004.
(60) Provisional application No. 60/652,246, filed on Feb. 11, 2005.

(51) Int. Cl. A61K 31/4184; C07D 403/02

(52) U.S. Cl. 514/394; 548/305.4

(57) ABSTRACT

Provided is the amorphous form of telmisartan sodium and the preparation thereof. Also provided are the telmisartan sodium polymorph crystal Forms 0 to XIII and XV to XX and preparations thereof. Also provided are pharmaceutical composition of amorphous and polymorphic forms of telmisartan sodium or mixtures thereof, and methods of treatment of a mammal in need thereof.

### RELATED APPLICATIONS

The present application claims the benefit of U.S. Provisional Patent Application No. 60/624,842 filed Nov. 3, 2004 and U.S. Provisional Patent Application No. 60/652,246 filed on Feb. 11, 2005, which are incorporated herein by reference.

### FIELD OF THE INVENTION

The present invention is directed to amorphous telmisartan sodium and processes for preparing such amorphous form of telmisartan sodium. The invention is also directed to polymorphic crystal structures of telmisartan sodium and to processes for preparing polymorphic crystal structures of telmisartan sodium. The invention is also directed to pharmaceutical compositions comprising polymorphic crystal structures and an amorphous form of telmisartan sodium.

### BACKGROUND OF THE INVENTION

Telmisartan is the common chemical name for the compound 4′-[2-n-propyl-4-methyl-6-(1-methylbenzimid-azol-2-yl)benzimidazol-1-ylmethyl]biphenyl-2-carboxylic acid. (CAS Registry No. 144701-48-4.) The empirical formula of telmisartan is C₃₃H₃₀N₄O₂ and its molecular weight is 514.63. The molecular structure of telmisartan is represented by Formula I.

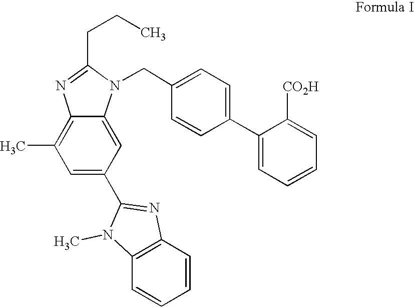

Telmisartan is a non-peptide angiotensin II receptor (type AT₁) antagonist. The United States Food and Drug Administration (FDA) approved it for the treatment of hypertension. It may be used alone or in combination with other hypertensive agents, such as hydrochlorothiazide. Boehringer Ingelheim markets telmisartan under the trade name Micardis® (telmisartan), available as 40 and 80 mg tablets for oral administration. Two patents are listed in the FDA's electronic Orange Book for telmisartan, U.S. Pat. No. 6,358,986 (“the '986 patent”) and U.S. Pat. No. 5,591,762 (“the '762 patent”).

The '986 patent discloses that telmisartan and the physiologically acceptable salts thereof can also be used to treat cardiac insufficiency, ischaemic peripheral circulatory disorders, myocardial ischaemia (angina), diabetic neuropathy, glaucoma, gastrointestinal diseases, bladder diseases, and to prevent progression of cardiac insufficiency after myocardial infarct.

In addition to the above therapeutic applications of telmisartan, the '762 patent discloses other therapeutic applications, including treating diabetic nephropathy, pulmonary diseases, e.g., lung oedema and chronic bronchitis. It also discloses using telmisartan to prevent arterial restenosis after angioplasty, thickening of blood vessel walls after vascular operations, and diabetic angiopathy. The '762 patent further discloses using telmisartan to alleviate central nervous system disorders, such as depression, Alzheimer's disease, Parkinson Syndrome, bulimia, and disorders of cognitive function in view of the effects of angiotensin on the release of acetylchloine and dopamine in the brain.

The European Application No. EP 0502314 and its corresponding U.S. patent, the '762 patent discloses preparing telmisartan by alkylation of 1,7′-dimethyl-2′-propyl-1H, 3′H-[2,5′] bibenzoimidazolyl (BIM) with 4′-[(bromomethyl)[1,1′-biphenyl]-2-carboxylic acid 1,1-dimethylethyl ester followed by hydrolysis.

The BIM has been prepared by mixing 2-propyl-4-methyl-1H-benzimidazole-6-carboxylic acid with N-methyl-o-phenylene-diamine or salts thereof, preferably in the form of the phosphate salt, in the presence of methanesulphonic acid and phosphorus pentoxide, as disclosed in J. Med. Chem. (1993), 36(25), 4040-51, International Patent Application WO 0063158, and US Application No. 2003/0139608.

Telmisartan alkyl-ester can be used as a starting material in the preparation of telmisartan polymorphs. In Chinese application no. CN 1344712, telmisartan methyl-ester was used for this purpose.

Many pharmaceutical solids can exist in different physical forms. Polymorphism is often characterized as the ability of a drug substance to exist as two or more crystalline phases that have different arrangements and/or conformations of the molecules in the crystalline lattice.

The present invention relates to the solid state physical properties of telmisartan sodium. These properties can be influenced by controlling the conditions under which telmisartan sodium is obtained in solid form. Solid state physical properties affect the ease with which the material is handled during processing into a pharmaceutical product such as a tablet or capsule formulation. The physical properties impact the sort of excipients, for instance, to add to a telmisartan sodium formulation. Furthermore, the solid state physical property of a pharmaceutical compound is important to its dissolution in aqueous fluid or even in a patient's stomach fluid, which have therapeutic consequences. The rate of dissolution is also a consideration in liquid forms of medicine as well. The solid state form of a compound may also affect its storage conditions.

These practical physical characteristics are influenced by the particular polymorphic form of a substance. One polymorphic form may give rise to thermal behavior different from that of the amorphous material or other polymorphic forms. Thermal behavior is measured in the laboratory by such techniques as capillary melting point, thermogravimetric analysis (TGA) and differential scanning calorimetry (DSC) and can be used to distinguish some polymorphic forms from others. A particular polymorphic form may also give rise to distinct spectroscopic properties that may be detectable by powder X-ray crystallography, solid state ¹³C NMR spectrometry and infrared spectrometry.

US 2003/0130331 discloses a crystalline sodium salt of telmisartan, characterized by x-ray powder diffraction peaks at 4.21, 4.98, 6.32 and 6.48 degrees two-theta±0.2 degrees two-theta, having a melting point of 245±5° C.

WO 04/028505 discloses a solid pharmaceutical composition comprising telmisartan, a basic agent, a surfactant or emulsifier, and a water-soluble diluent, e.g. in the form of granules or powder for use in e.g. capsule or tablet formulations and methods for producing them using a fluid-bed granulation process or spray-drying process.

The present invention relates to amorphous and polymorphic forms of telmisartan sodium. Moreover the present invention relates to additional processes for preparation of amorphous and polymorphic forms of telmisartan sodium

### SUMMARY OF THE INVENTION

In one aspect, the present invention provides an amorphous form of telmisartan sodium.

In another aspect, the present invention provides crystalline telmisartan sodium, herein defined as Form 0, characterized by X-ray powder diffraction peaks at 2.4, 4.7, 6.2, 7.1, 7.4, 15.3, and 22.3 degrees two-theta±0.2 degrees two-theta.

In another aspect, the present invention provides crystalline telmisartan sodium, herein defined as Form I, characterized by X-ray powder diffraction peaks at 4.1, 5.0, 6.2, 7.0, 7.4, and 15.2 degrees two-theta±0.2 degrees two-theta.

In another aspect, the present invention provides crystalline telmisartan sodium, herein defined as Form II, characterized by X-ray powder diffraction peaks at 4.6, 7.4, 7.7, 15.3, and 22.4 degrees two-theta±0.2 degrees two-theta.

In another aspect, the present invention provides crystalline telmisartan sodium, herein defined as Form III, characterized by X-ray powder diffraction peaks at 4.0, 4.3, and 5.1 degrees two-theta±0.2 degrees two-theta.

In another aspect, the present invention provides crystalline telmisartan sodium, herein defined as Form IV, characterized by X-ray powder diffraction peaks at 4.5, 4.9, 7.3, 15.2, 19.3, and 22.4 degrees two-theta±0.2 degrees two-theta.

In another aspect, the present invention provides crystalline telmisartan sodium, herein defined as Form V, characterized by X-ray powder diffraction peaks at 4.1, 5.2, 5.6, 7.6, 8.0, 8.4, 11.9, 15.9, 19.7, and 23.2 degrees two-theta±0.2 degrees two-theta.

In another aspect, the present invention provides crystalline telmisartan sodium, herein defined as Form VI, characterized by X-ray powder diffraction peaks at 6.0, 6.8, 10.4, 11.8, 16.7, 17.7, 19.3, 20.8, and 23.5 degrees two-theta±0.2 degrees two-theta.

In another aspect, the present invention provides crystalline telmisartan sodium, herein defined as Form VII, characterized by X-ray powder diffraction peaks at 5.6, 10.0, 14.8, 17.4, and 20.4 degrees two-theta±0.2 degrees two-theta.

In another aspect, the present invention provides crystalline telmisartan sodium, herein defined as Form VIII, characterized by X-ray powder diffraction peaks at 4.0, 4.4, 6.3, and 6.8 degrees two-theta±0.2 degrees two-theta.

In another aspect, the present invention provides crystalline telmisartan sodium, herein defined as Form IX, characterized by X-ray powder diffraction peaks at 3.7, 4.6, 6.2, and 16.4 degrees two-theta±0.2 degrees two-theta.

In another aspect, the present invention provides crystalline telmisartan sodium, herein defined as Form X, characterized by X-ray powder diffraction peaks at 3.6, 6.1, 15.0, 17.6,20.7, and 22.1 degrees two-theta±0.2 degrees two-theta.

In another aspect, the present invention provides crystalline telmisartan sodium, herein defined as Form XI, characterized by X-ray powder diffraction peaks at 4.2, 4.5, and 5.5 degrees two-theta±0.2 degrees two-theta.

In another aspect, the present invention provides crystalline telmisartan sodium, herein defined as Form XII, characterized by X-ray powder diffraction peaks at 3.5, 6.0, 6.9, 16.1, 19.5, and 23.0 degrees two-theta±0.2 degrees two-theta.

In another aspect, the present invention provides crystalline telmisartan sodium, herein defined as Form XIII, characterized by X-ray powder diffraction peaks at 3.9, 4.6, 6.0, 6.3, and 6.8 degrees two-theta±0.2 degrees two-theta.

In another aspect, the present invention provides crystalline telmisartan sodium, herein defined as Form XV, characterized by X-ray powder diffraction peaks at 4.3, 5.0, 6.9, 8.6, and 16.0 degrees two-theta±0.2 degrees two-theta.

In another aspect, the present invention provides crystalline telmisartan sodium, herein defined as Form XVI, characterized by X-ray powder diffraction peaks at 4.0, 5.6, 6.8, 10.3, 12.3, 16.8, and 17.5 degrees two-theta±0.2 degrees two-theta.

In another aspect, the present invention provides crystalline telmisartan sodium, herein defined as Form XVII, characterized by X-ray powder diffraction peaks at 4.1, 4.9, 5.3, 8.1, 11.6, 15.2, and 20.7 degrees two-theta±0.2 degrees two-theta.

In another aspect, the present invention provides crystalline telmisartan sodium, herein defined as Form XVIII, characterized by X-ray powder diffraction peaks at 4.7, 5.0, 6.3, and 6.8 degrees two-theta±0.2 degrees two-theta.

In another aspect, the present invention provides crystalline telmisartan sodium, herein defined as Form XIX, characterized by X-ray powder diffraction peaks at 4.3, 4.7, 6.9, and 7.5 degrees two-theta±0.2 degrees two-theta.

In another aspect, the present invention provides crystalline telmisartan sodium, herein defined as Form XX, characterized by X-ray powder diffraction peaks at 3.5, 6.0, 6.9, 15.6, 19.0, 20.6, and 22.4 degrees two-theta±0.2 degrees two-theta.

In another aspect, the present invention provides methods of preparing amorphous and polymorphic forms of telmisartan sodium In another aspect, the present invention provides pharmaceutical compositions of amorphous and polymorphic forms of telmisartan sodium or mixtures thereof, and methods of treatment of a mammal in need thereof.

### BRIEF DESCRIPTION OF THE FIGURES

FIG. 1 is a representative X-ray diffraction pattern of amorphous Telmisartan Sodium.

FIG. 2 illustrates the X-Ray Powder Diffraction pattern of Telmisartan Sodium Form 0.

FIG. 3 illustrates the X-Ray Powder Diffraction pattern of Telmisartan Sodium Form I.

FIG. 4 illustrates the X-Ray Powder Diffraction pattern of Telmisartan Sodium Form II.

FIG. 5 illustrates the X-Ray Powder Diffraction pattern of Telmisartan Sodium Form III.

FIG. 6 illustrates the X-Ray Powder Diffraction pattern of Telmisartan Sodium Form IV.

FIG. 7 illustrates the X-Ray Powder Diffraction pattern of Telmisartan Sodium Form V.

FIG. 8 illustrates the X-Ray Powder Diffraction pattern of Telmisartan Sodium Form VI.

FIG. 9 illustrates the X-Ray Powder Diffraction pattern of Telmisartan Sodium Form VII.

FIG. 10 illustrates the X-Ray Powder Diffraction pattern of Telmisartan Sodium Form VIII.

FIG. 11 illustrates the X-Ray Powder Diffraction pattern of Telmisartan Sodium Form IX.

FIG. 12 illustrates the X-Ray Powder Diffraction pattern of Telmisartan Sodium Form X.

FIG. 13 illustrates the X-Ray Powder Diffraction pattern of Telmisartan Sodium Form XI.

FIG. 14 illustrates the X-Ray Powder Diffraction pattern of Telmisartan Sodium Form XII.

FIG. 15 illustrates the X-Ray Powder Diffraction pattern of Telmisartan Sodium Form XIII.

FIG. 16 illustrates the X-Ray Powder Diffraction pattern of Telmisartan Sodium Form XV.

FIG. 17 illustrates the X-Ray Powder Diffraction pattern of Telmisartan Sodium Form XVI.

FIG. 18 illustrates the X-Ray Powder Diffraction pattern of Telmisartan Sodium Form XVII.

FIG. 19 illustrates the X-Ray Powder Diffraction pattern of Telmisartan Sodium Form XVIII.

FIG. 20 illustrates the X-Ray Powder Diffraction pattern of Telmisartan Sodium Form XIX.

FIG. 21 illustrates the X-Ray Powder Diffraction pattern of Telmisartan Sodium Form XX.

FIG. 22 illustrates the DSC thermogram of Telmisartan Sodium in amorphous form.

FIG. 23 illustrates the DSC thermogram of Telmisartan Sodium Form 0.

FIG. 24 illustrates the DSC thermogram of Telmisartan Sodium Form I.

FIG. 25 illustrates the DSC thermogram of Telmisartan Sodium Form II.

FIG. 26 illustrates the DSC thermogram of Telmisartan Sodium Form III.

FIG. 27 illustrates the DSC thermogram of Telmisartan Sodium Form IV.

FIG. 28 illustrates the DSC thermogram of Telmisartan Sodium Form V.

FIG. 29 illustrates the DSC thermogram of Telmisartan Sodium Form VI.

FIG. 30 illustrates the DSC thermogram of Telmisartan Sodium Form VII.

FIG. 31 illustrates the DSC thermogram of Telmisartan Sodium Form VIII.

FIG. 32 illustrates the DSC thermogram of Telmisartan Sodium Form IX.

FIG. 33 illustrates the DSC thermogram of Telmisartan Sodium Form X.

FIG. 34 illustrates the DSC thermogram of Telmisartan Sodium Form XI.

FIG. 35 illustrates the DSC thermogram of Telmisartan Sodium Form XII.

FIG. 36 illustrates the DSC thermogram of Telmisartan Sodium Form XIII.

FIG. 37 illustrates the DSC thermogram of Telmisartan Sodium Form XV.

FIG. 38 illustrates the DSC thermogram of Telmisartan Sodium Form XVI.

FIG. 39 illustrates the DSC thermogram of Telmisartan Sodium Form XVII.

FIG. 40 illustrates the DSC thermogram of Telmisartan Sodium Form XVIII.

FIG. 41 illustrates the DSC thermogram of Telmisartan Sodium Form XIX.

FIG. 42 illustrates the DSC thermogram of Telmisartan Sodium Form XX.

### DETAILED DESCRIPTION OF THE INVENTION

As used herein, the abbreviation TLM-Me refers to telmisartan methyl-ester.

As used herein amorphous form refers to a solid state where the solid contains less than about 20% crystalline telmisartan, more preferably less than about 10% and most preferably less than about 1% weight to weight. Amorphous solids consist of disordered arrangements of molecules and do not possess a distiguishable crystal lattice. An amorphous solid generally is more soluble than its crystalline form, leading to a more rapid bioavailability. Lack of peaks in a powder XRD pattern or lack of a endothermic melting peak in a DSC thermogram may indicate presence of an amorphous form. The area under the peaks in an XRD pattern may be added to obtain total amount of crystalline material. In a DSC thermogram the presence of endotherms may point to the melting of crystalline material.

As used herein, an anti-solvent is a liquid that when added to a solution of telmisartan sodium in a solvent, induces precipitation of telmisartan sodium. Precipitation of telmisartan sodium is induced by the anti-solvent when addition of the anti-solvent causes telmisartan sodium to precipitate from the solution, or to precipitate more rapidly, or to precipitate to a greater extent than telmisartan sodium would precipitate out of the solvent without the anti-solvent.

As used herein, precipitation can be perceived visually as cloudiness of the solution or formation of distinct particles of the various crystal forms of telmisartan sodium suspended in the solution or recovered at the bottom the vessel containing the solution.

As used herein drying refers to removal of solvent or solution from a solid, which can be achieved by applying heat or applying a temperature that is about room temperature at a pressure below about 100 mmHg in a vacuum oven.

As used herein, “room temperature” refers to a temperature of from about 20° C. to about 25° C.

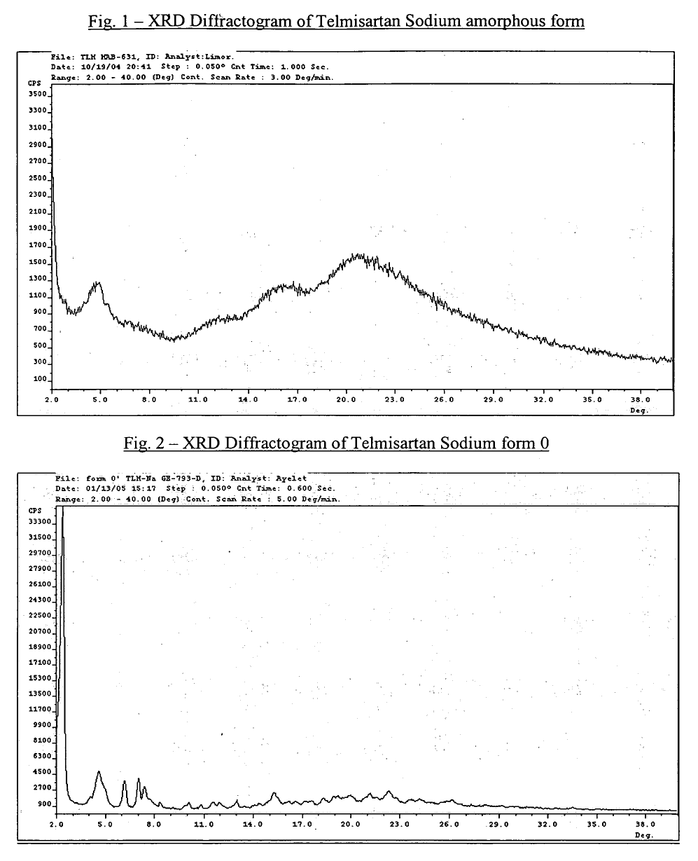

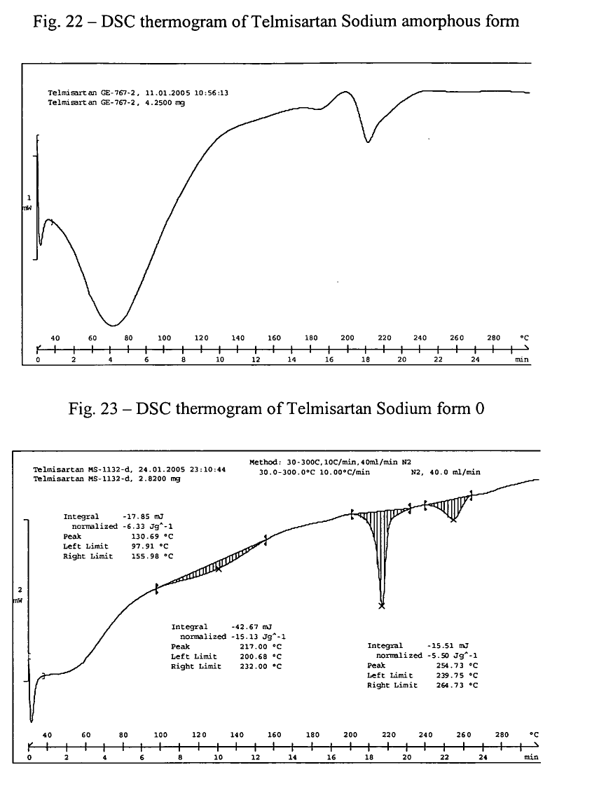

In one embodiment, the present invention provides an amorphous form of telmisartan sodium. Amorphous telmisartan sodium has an X-ray diffraction pattern typical for an essentially amorphous solid. The X-ray diffraction of amorphous telmisartan sodium shows a halo-pattern lacking visible crystalline peaks. FIG. 1 shows a representative x-ray diffraction diagram of amorphous telmisartan sodium. Further, FIG. 22 shows a representative thermogram from differential scanning calorimetry (DSC) for amorphous telmisartan sodium. The DSC thermogram does not exhibit any feature that can be clearly associated with a first-order transition like the melting of a crystal.

In another embodiment, the present invention provides a method of preparing amorphous telmisartan sodium comprising: providing a mixture of telmisartan C₁-C₄ alkylester, a first solvent and an inorganic base; heating the mixture to a temperature of from about 65° C. to about 90° C.; combining the mixture with a second solvent and removing the solvents to obtain amorphous telmisartan sodium.

Preferably, the C₁-C₄ alkyl-ester group in the telmisartan C₁-C₄ alkyl-ester starting material is selected from the group consisting of methyl-ester, ethyl-ester, isopropyl-ester, propyl-ester, butyl-ester and t-butyl-ester. More preferably, the starting material is telmisartan methyl-ester.

Preferably, the first solvent is selected from a group consisting of a C₁₋₄ alcohol, C₃-C₇ ketone or tetrahydrofuran. More preferably, the first solvent is selected from a group consisting of ethanol, methyl ethyl ketone or tetrahydrofuran. Most preferably, the first solvent is ethanol.

Preferably, the inorganic base is an alkaline metal hydroxide or an alkaline earth metal hydroxide. More preferably, the inorganic base is selected from a group consisting of potassium hydroxide, sodium hydroxide, lithium hydroxide, magnesium hydroxide and calcium hydroxide. Most preferably, the inorganic base is sodium hydroxide so that a sodium salt is immediately formed.

The mixture is heated to accelerate the hydrolysis of the telmisartan alkylester to a telmisartan sodium.

Preferably, the mixture is heated to about the reflux temperature of the solvent, preferably, for more than two hours. More preferably, for about 2 hours to about 24 hours. Most preferably, for about 4 hours to about 8 hours.

Preferably, the second solvent is selected from a group consisting of water, a C₁-C₄ alcohol, C₃-C₇ ketone or tetrahydrofuran. More preferably, the first solvent is selected from a group consisting of water, ethanol, methyl ethyl ketone or tetrahydrofuran, Most preferably, the organic solvent is water or ethanol.

Since organic solvents, particularly alcohols, generally have a lower freezing point than water, an organic solvent, particularly technical grade (less than 2% water by volume) is preferably removed by evaporation without freezing of the solvent. The solvent may be removed under a pressure below one atmosphere to accelerate the process, such as a pressure below about 100 mmHg. The evaporation may be carried out by injecting the solution into a vacuum chamber, so that the solvent readily evaporates, causing formation of an amorphous form before crystal formation. The chamber may be heated to a temperature of from about room temperature to about 50° C. Preferably for about 40° C.

Preferably, if the second solvent is water, after evaporating the first solvent, the solution is lyophilized.

The lyophilization may be preformed under various combinations of temperature and pressure that result in vaporization of a frozen solvent. In one embodiment, lyophilization is performed by maintaining the aqueous mixture at a temperature of about −40° C. to about −170° C. and at a pressure of about 0.2 to about 0.5 mm Hg for a sufficient time, preferably a period of about 16 hours to about one week. Preferably, the lyophilization is performed at a temperature of about −50° C. for a period of about 4 days.

In another embodiment, the present invention provides a method of preparing amorphous telmisartan sodium comprising; providing a mixture of telmisartan, a polar organic solvent and an inorganic base; maintaining the mixture at room temperature and removing the solvents to obtain amorphous telmisartan sodium.

The method above can also include filtering the solution before evaporating the solvents.

The obtained mixture can be either a solution or a slurry.

Preferably, the polar organic solvent is selected from a group consisting of: a C₁-C₄ alcohol, C₃-C₇ ketone and tetrahydrofuran. More preferably, the polar organic solvent is selected from a group consisting of: ethanol, methyl ethyl ketone and tetrahydrofuran. Most preferably, the polar organic solvent is ethanol.

Preferably, the inorganic base is as described above.

Preferably, the mixture is maintained for at least half an hour. More preferably, for about half an hour to about 7 hours. Most preferably, for about 3.5 hours.

Preferably, the solvents are removed by evaporation, as described above.

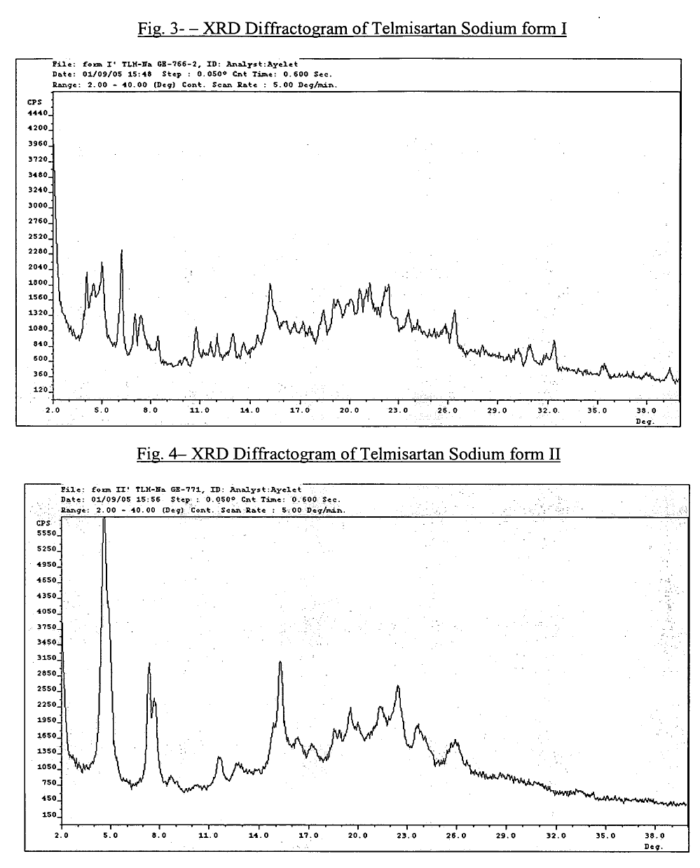

Another embodiment of the invention provides a crystalline form of telmisartan sodium, herein defined as Form 0, which is characterized by an X-ray powder diffraction pattern with peaks at 2.4, 4.7, 6.2, 7.1, 7.4, 15.3, and 22.3 degrees two-theta±0.2 degrees two-theta, substantially as depicted in FIG. 2. Form 0 may also be identified by a Differential scanning calorimetry thermogram with peaks at about 131, 217, and 255° C., substantially as depicted in FIG. 23.

Another embodiment of the present invention provides a process for preparing telmisartan sodium crystal Form 0 comprising heating a solution of telmisartan sodium in ethanol, preferably heating the solution at a temperature of about 65° C. to 90° C., more preferably heating the solution to reflux; combining ethylacetate with the heated solution forming a mixture; adding additional ethylacetate to the mixture; allowing the mixture to precipitate; and recovering the precipitate. Preferably, allowing the mixture to precipitate comprises allowing the mixture to cool to about 20-40° C. while agitating, such as stirring, the mixture, more preferably the mixture is cooled to room temperature. Preferably, recovering the precipitate is performed by filtration; more preferably filtration at a pressure below about 100 mmHg, vacuum filtration. The process may further comprise drying the precipitate, more preferably, the precipitate is dried at about room temperature at a pressure below about 100 mmHg in a vacuum oven to obtain telmisartan sodium Form 0.

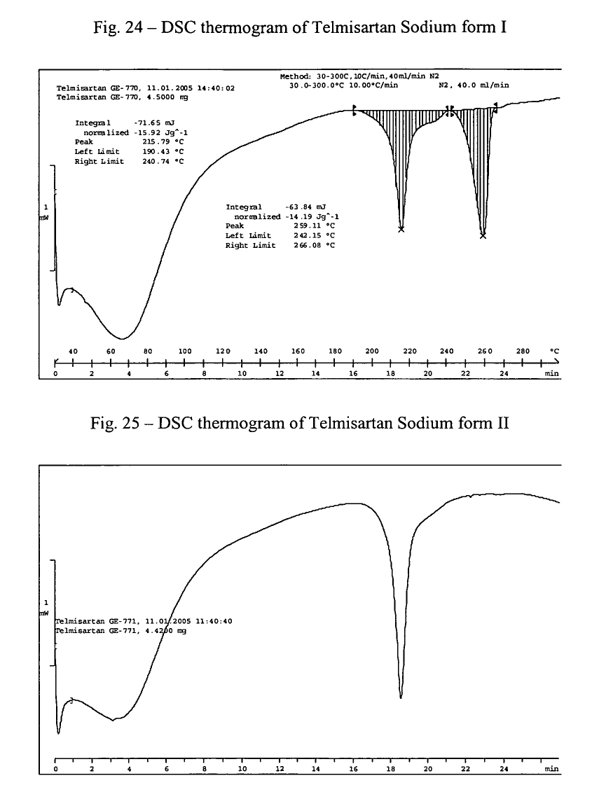

Another embodiment of the invention provides a crystalline form of telmisartan sodium, herein defined as Form I, which is characterized by an X-ray powder diffraction pattern with peaks at 4.1, 5.0, 6.2, 7.0, 7.4, and 15.2 degrees two-theta±0.2 degrees two-theta, substantially as depicted in FIG. 3. Form I may also be identified by a Differential scanning calorimetry thermogram with peaks at about 216 and 259° C., substantially as depicted in FIG. 24.

Another embodiment of the present invention provides a process for preparing telmisartan sodium Form I comprising dissolving telmisartan sodium in ethanol forming a solution; heating the solution, preferably heating the solution at a temperature of about 65° C. to 90° C., more preferably heating the solution to reflux; combining hexane with the heated solution forming a mixture; allowing the mixture to precipitate; and recovering the precipitate. Preferably, allowing the mixture to precipitate comprises allowing the mixture to cool to about 20-40° C., preferably the mixture is cooled to room temperature. Preferably, the precipitate is recovered by filtration, more preferably filtered at a pressure below about 100 mmHg by vacuum filtration. Preferably, the precipitate is washed with solvent, more preferably the precipitate is washed with hexane. The process may further comprise drying the precipitate, preferably the precipitate is dried at about room temperature at a pressure below about 100 mmHg in a vacuum oven to obtain telmisartan sodium Form I.

Yet another embodiment of the present invention provides a process for preparing telmisartan sodium crystal Form I comprising dissolving telmisartan sodium in isopropyl alcohol to form a solution; heating the solution, preferably heating the solution at a temperature of about 70° C. to 95° C., more preferably heating the solution to reflux; cooling the heated solution to about 20-40° C.; combining methyl tertiary butyl ether (MTBE) with the cooled solution forming a sticky precipitate; reheating the sticky precipitate solution, preferably reheating the solution at a temperature of about 45° C. to 65° C., more preferably reheating the solution to reflux; combining isopropyl alcohol with the reheated solution forming a homogeneous slurry; allowing the mixture to precipitate; and recovering the precipitate. Preferably allowing the slurry to precipitate comprises allowing the slurry to cool to about 20-40° C., more preferably the slurry is cooled to room temperature. Preferably the precipitate is recovered by filtration, more preferably at a pressure below about 100 mmHg by vacuum filtration. Preferably, the precipitate is washed with solvent, more preferably the precipitate is washed with MTBE. The process may further comprise drying the precipitate, preferably the precipitate is dried at about room temperature at a pressure below about 100 mmHg in a vacuum oven to obtain telmisartan sodium form I.

Yet another embodiment of the invention provides a crystalline form of telmisartan sodium, herein defined as Form II, which is characterized by an X-ray powder diffraction pattern with peaks at 4.6, 7.4, 7.7, 15.3, and 22.4 degrees two-theta±0.2 degrees two-theta, substantially as depicted in FIG. 4. Form II may also be identified by its characteristic differential scanning calorimetry thermogram, substantially as depicted in FIG. 25.

Another embodiment of the invention provides a process for preparing telmisartan sodium crystal Form I and II comprising dissolving telmisartan sodium in isopropyl alcohol forming a solution and heating the solution, preferably heating the solution at a temperature of about 70° C. to 95° C., more preferably heating to reflux; combining MTBE with the solution forming a mixture; allowing the mixture to precipitate; and recovering the precipitate. Preferably, allowing the mixture to precipitate comprises allowing the mixture to cool to about 20-40° C., more preferably cooling to about room temperature. Preferably, the precipitate is recovered by filtration, more preferably at a pressure below about 100 mmHg by vacuum filtration. Preferably, the precipitate is washed with solvent, more preferably with MTBE. The process may further comprise drying the precipitate, preferably drying the precipitate at about room temperature at a pressure below about 100 mmHg in a vacuum oven to obtain telmisartan sodium Form I and II.

Another embodiment of the invention provides a process for preparing telmisartan sodium crystal Form II comprising dissolving telmisartan sodium in ethanol forming a solution; heating the solution, preferably heating the solution at a temperature of about 65° C. to 90° C., more preferably heating the solution to reflux; combining MTBE with the heated solution and allowing the solution to precipitate; and recovering the precipitate. Preferably, allowing the solution to precipitate comprises allowing the solution to cool to about 20-40° C., more preferably, cooling to about room temperature. Preferably, the precipitate is recovered by filtration; more preferably at a pressure below about 100 mmHg by vacuum filtration. Preferably, the precipitate is washed with solvent, more preferably with MTBE. The process may further comprise drying the precipitate to obtain telmisartan sodium Form II.

Yet another embodiment of the present invention provides a process for preparing telmisartan sodium mixture of crystal Form I and crystal Form II comprising dissolving telmisartan sodium in ethanol to form a solution; heating the solution, preferably heating the solution at a temperature of about 65° C. to 90° C., more preferably to reflux; combining diethyl-ether with the heated solution; allowing the solution to precipitate; and recovering the precipitate. Preferably, allowing the solution to precipitate comprises allowing the solution to cool to about 20-40° C. Preferably, the precipitate is recovered by filtration, more preferably at a pressure below about 100 mmHg by vacuum filtration. Preferably, the precipitate is washed with solvent, more preferably the precipitate is washed with diethyl-ether. The process may further comprise drying the precipitate to obtain telmisartan sodium crystal Form I and II.

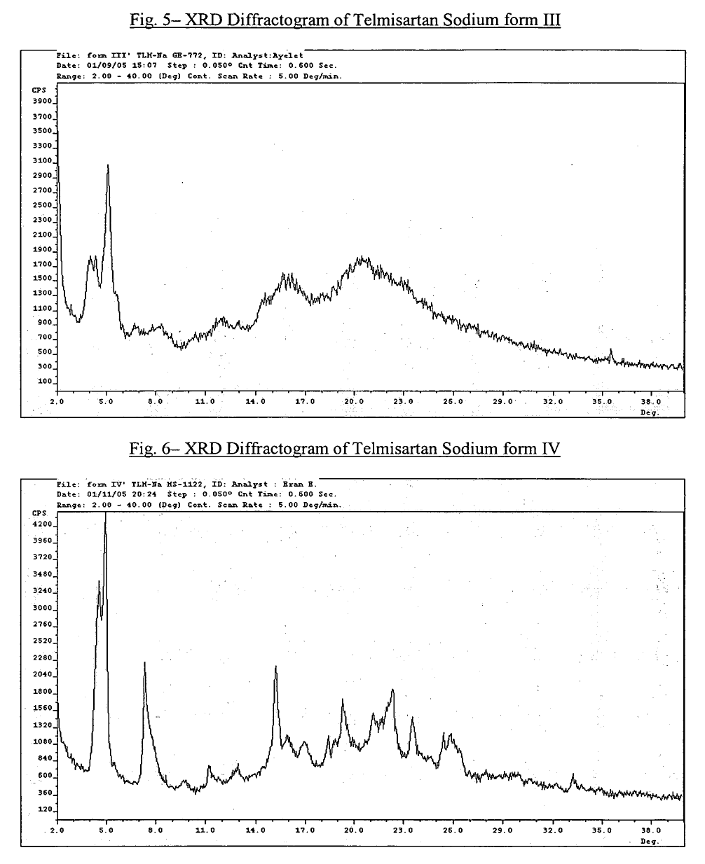

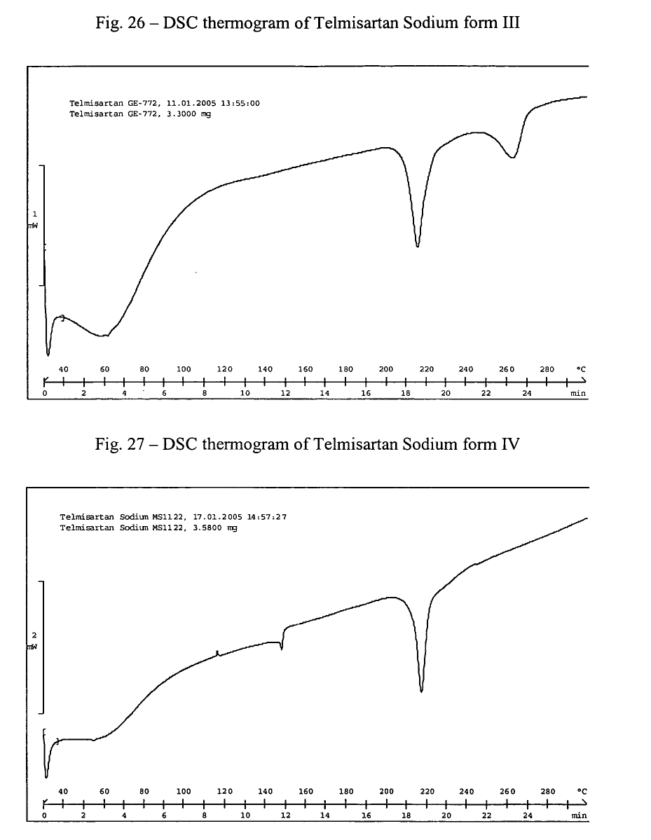

Another embodiment of the invention provides a crystalline form of telmisartan sodium, herein defined as Form III, which is characterized by an X-ray powder diffraction pattern with peaks at 4.0, 4.3, and 5.1 degrees two-theta±0.2 degrees two-theta, substantially as depicted in FIG. 5. Form III may also be identified by its characteristic differential scanning calorimetry thermogram, substantially as depicted in FIG. 26.

Another embodiment of the invention provides a process for preparing telmisartan sodium crystal Form III comprising dissolving telmisartan sodium in methanol forming a solution; the solution is heated, preferably at a temperature of about 55° C. to 75° C., more preferably to reflux; combining MTBE with the heated solution forming a mixture; allowing the solution to precipitate; and recovering the precipitate. Preferably, allowing the solution to precipitate comprises allowing the mixture to cool to about 20-40° C. Preferably, the precipitate is recovered by filtration, more preferably at a pressure below about 100 mmHg by vacuum filtration. The process may further comprise drying the precipitate to obtain telmisartan sodium Form III.

Another embodiment of the invention provides a crystalline form of telmisartan sodium, herein defined as Form IV, which is characterized by an X-ray powder diffraction pattern with peaks at 4.5, 4.9, 7.3, 15.2, 19.3, and 22.4 degrees two-theta±0.2 degrees two-theta, substantially as depicted in FIG. 6. Form IV may also be identified by its characteristic differential scanning calorimetry thermogram, substantially as depicted in FIG. 27.

Yet another embodiment of the invention provides a process for preparing telmisartan crystal Form IV comprising drying telmisartan sodium crystal Form XI to form telmisartan sodium crystal Form IV product.

Another embodiment of the invention provides a process for preparing telmisartan crystal Form IV comprising dissolving telmisartan in methanol and sodium hydroxide forming a solution; filtering the solution; evaporating the solution forming a residue; dissolving the residue in ethanol by heating, preferably heating the solution at a temperature of about 65° C. to 90° C., more preferably to reflux; combining MTBE with the heated solution; allowing the solution to precipitate; and recovering the precipitate. Preferably, allowing the solution to precipitate comprises allowing the solution to cool to about 20-40° C., more preferably, cooling to about room temperature. Preferably, the precipitate is recovered by filtration, more preferably at a pressure below about 100 mmHg by vacuum filtration. Preferably, the precipitate is washed with solvent, more preferably with MTBE. The process may further comprise drying the precipitate to obtain telmisartan sodium crystal Form IV.

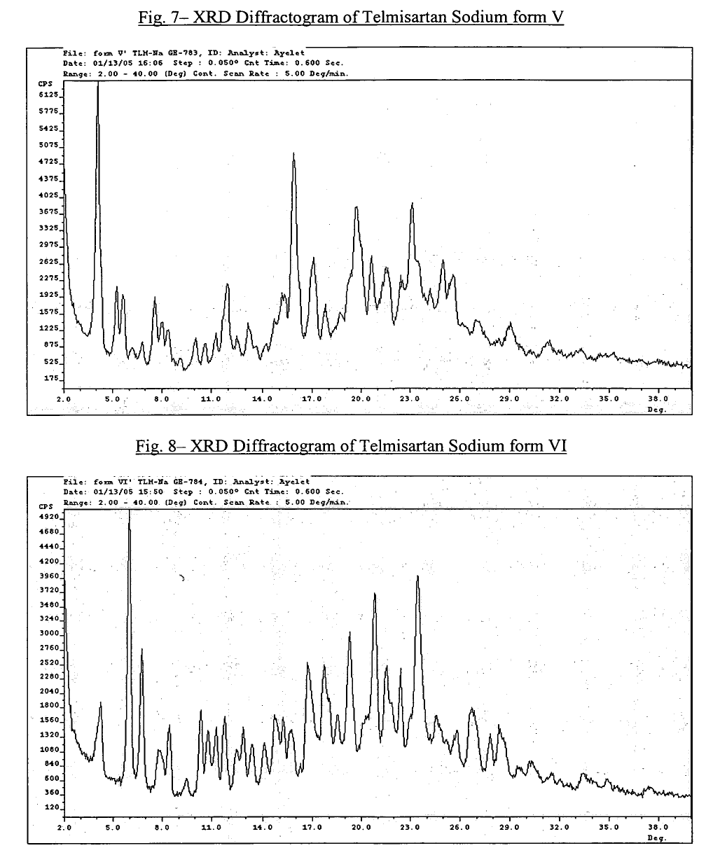

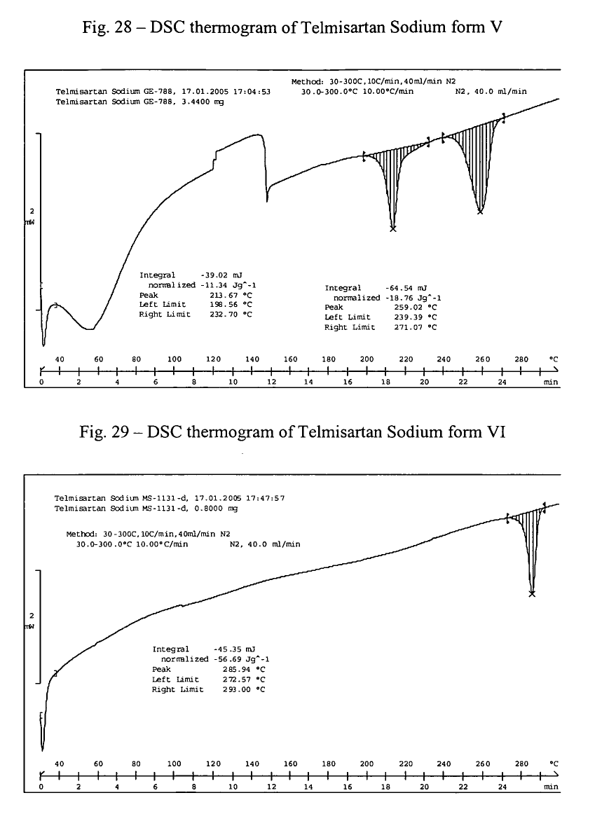

Yet another embodiment of the invention provides a crystalline form of telmisartan sodium, herein defined as Form V, which is characterized by an X-ray powder diffraction pattern with peaks at 4.1, 5.2, 5.6, 7.6, 8.0, 8.4, 11.9, 15.9, 19.7, and 23.2 degrees two-theta±0.2 degrees two-theta, substantially as depicted in FIG. 7. Form V may also be identified by a differential scanning calorimetry thermogram with peaks at about 214 and 259° C., substantially as depicted in FIG. 28.

Another embodiment of the invention provides a process for preparing telmisartan sodium crystal Form V comprising heating telmisartan sodium crystal Form IV in toluene, at a temperature of about 95° C. to 125° C., preferably to reflux, forming a slurry; allowing the slurry to precipitate; and recovering the precipitate. Preferably, allowing the slurry to precipitate comprises allowing the slurry to cool to about 20-40° C., more preferably, cooling to about room temperature. Preferably, the precipitate is recovered by filtration, more preferably at a pressure below about 100 mmHg by vacuum filtration. Preferably, the precipitate is washed with solvent, more preferably with toluene. The process may further comprise drying the washed precipitate, preferably drying the precipitate at about room temperature at a pressure below about 100 mmHg in a vacuum oven to obtain telmisartan sodium crystal Form V product.

Another embodiment of the invention provides a process for preparing telmisartan sodium crystal Form V comprising heating a mixture of amorphous telmisartan sodium in toluene at temperature of about 95° C. to 125° C., preferably heating the mixture to reflux in toluene, forming a slurry; allowing the slurry to precipitate and recovering the precipitate. Preferably, allowing the slurry to precipitate comprises allowing the solution to cool to about 20-40° C., more preferably cooling to room temperature. Preferably, the precipitate is recovered filtration, more preferably at a pressure below about 100 mmHg by vacuum filtration. Preferably, the precipitate is washed with solvent, more preferably with toluene. The process may further comprise drying the washed filtrate at about room temperature at a pressure below about 100 mmHg in a vacuum oven to obtain telmisartan sodium crystal Form V.

Another embodiment of the invention provides a crystalline form of telmisartan sodium, herein defined as Form VI, which is characterized by an X-ray powder diffraction pattern with peaks at 6.0, 6.8, 10.4, 11.8, 16.7, 17.7, 19.3, 20.8, and 23.5 degrees two-theta±0.2 degrees two-theta, substantially as depicted in FIG. 8. Form VI may also be identified by a Differential scanning calorimetry thermogram with peaks at about 286° C., substantially as depicted in FIG. 29.

Another embodiment of the invention provides a process for preparing telmisartan sodium crystal Form VI comprising heating a mixture of telmisartan sodium crystal Form IV and methyl ethyl ketone at a temperature of about 65° C. to 95° C., preferably heating to reflux, forming a slurry; allowing the slurry to precipitate and recovering the precipitate. Preferably, allowing the slurry to precipitate comprises allowing the slurry to cool to about 20-40° C., while stirring. Preferably, recovering the precipitate is performed by filtration, more preferably at about room temperature at a pressure below about 100 mmHg by vacuum filtration. Preferably, the precipitate is washed with solvent, more preferably with methyl ethyl ketone. The process may further comprise drying the washed filtrate at about room temperature at a pressure below about 100 mmHg in a vacuum to obtain telmisartan sodium crystal Form VI.

Yet another embodiment of the invention provides a process for preparing telmisartan crystal Form VI comprising heating a solution of telmisartan sodium in ethanol at a temperature of about 65° C. to 90° C., preferably heating telmisartan sodium to reflux forming a solution; combining acetonitrile with the solution forming a mixture; allowing the mixture to precipitate; and recovering the precipitate. Preferably, allowing the solution to precipitate comprises allowing the solution to cool to about 20-40° C.; while stirring. Preferably, recovering the precipitate is performed by filtration, more preferably at a pressure below about 100 mmHg by vacuum filtration. Preferably, the precipitate is washed with solvent, more preferably with acetonitrile. The process may further comprise drying the washed filtrate at about room temperature at a pressure below about 100 mmHg in a vacuum oven in a vacuum oven to obtain telmisartan sodium crystal Form VI.

Another embodiment of the invention provides a process for preparing telmisartan crystal Form VI comprising drying telmisartan sodium crystal Form XII, preferably at about room temperature at a pressure below about 100 mmHg in a vacuum oven to obtain telmisartan sodium crystal Form VI.

Another embodiment of the invention provides a process for preparing telmisartan crystal Form VI comprising drying telmisartan sodium crystal Form XX, preferably at about room temperature at a pressure below about 100 mmHg in a vacuum oven to obtain telmisartan sodium crystal Form VI.

Yet another embodiment of the invention provides a process for preparing telmisartan crystal Form VI comprising drying wet telmisartan sodium Form X at about room temperature at a pressure below about 100 mmHg in a vacuum oven to obtain telmisartan sodium crystal Form VI product.

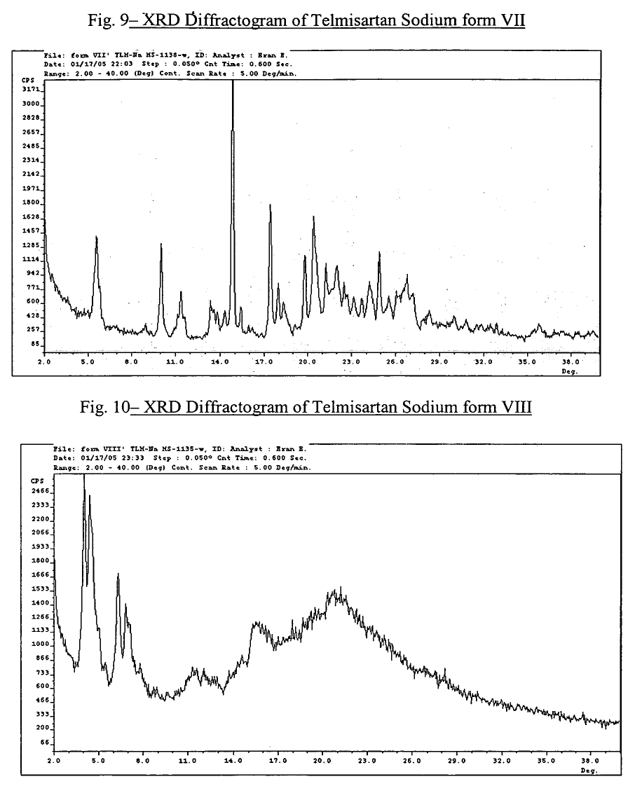

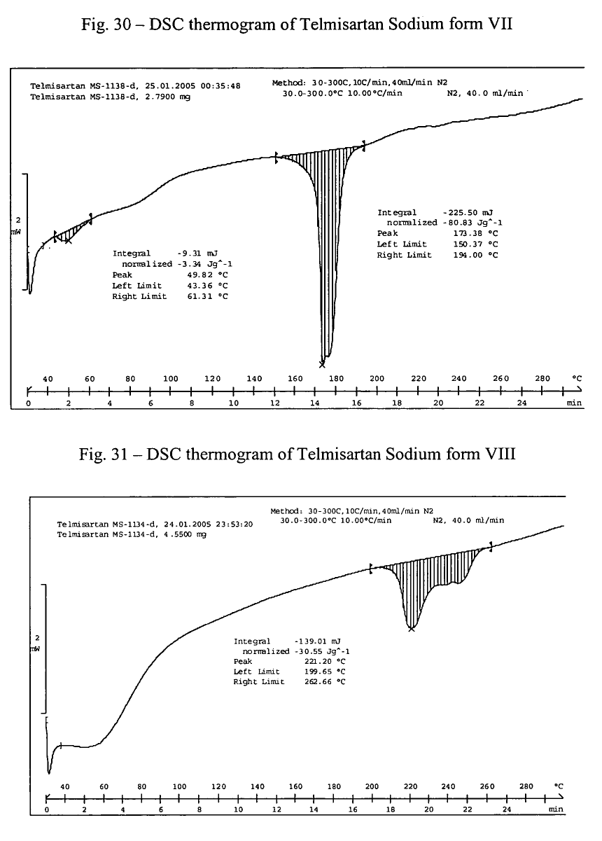

Another embodiment of the invention provides a crystalline form of telmisartan sodium, herein defined as Form VII, which is characterized by an X-ray powder diffraction pattern with peaks at 5.6, 10.0, 14.8, 17.4, and 20.4 degrees two-theta±0.2 degrees two-theta, substantially as depicted in FIG. 9. Form VII may also be identified by a differential scanning calorimetry thermogram with peaks at about 50 and 173° C., substantially as depicted in FIG. 30.

Another embodiment of the invention provides a process for preparing telmisartan sodium crystal form VII comprising heating telmisartin sodium in dimethylsulfoxide (DMSO) forming a solution, preferably heating the solution to a temperature of from about 75° C. to about 95° C., more preferably to about 90° C.; combining ethylacetate with the heated solution forming an heated mixture; allowing the solution to precipitate; and recovering the precipitate. Preferably, allowing the solution to precipitate comprises allowing the solution to cool to about 20-40° C.; and adding ethylacetate while stirring. Preferably, recovering the precipitate is performed by filtration, more preferably at a pressure below about 100 mmHg by vacuum filtration. Preferably, the precipitate is washed with solvent, more preferably with ethylacetate. The process may further comprise drying the washed filtrate at about room temperature at a pressure below about 100 mmHg in a vacuum oven to obtain telmisartan sodium crystal Form VII.

Yet another embodiment of the invention provides a crystalline form of telmisartan sodium, herein defined as Form VIII, which is characterized by an X-ray powder diffraction pattern with peaks at 4.0, 4.4, 6.3, and 6.8 degrees two-theta±0.2 degrees two-theta, substantially as depicted in FIG. 10. Form VIII may also be identified by a differential scanning calorimetry thermogram with peaks at about 221° C., substantially as depicted in FIG. 31.

Another embodiment of the invention provides a process for preparing telmisartan sodium crystal Form VIII comprising heating a solution of telmisartan sodium in dichloromethane at a temperature of about 30° C. to 55° C., preferably heating to reflux, forming a solution; combining MTBE with the solution forming a mixture; allowing the solution to precipitate; and recovering the precipitate. Preferably, allowing the solution to precipitate comprises allowing the solution to cool to about 20-40° C. Preferably, recovering the precipitate is performed by filtration, more preferably at about room temperature at a pressure below about 100 mmHg by vacuum filtration. Preferably, the precipitate is washed with solvent, more preferably with MTBE, to obtain wet telmisartan sodium Form VIII. The process may further comprise drying the wet telmisartan sodium Form VIII at about room temperature at a pressure below about 100 mmHg in a vacuum oven to obtain telmisartan sodium Form VIII.

Yet another embodiment of the invention provides a process for preparing telmisartan sodium crystal Form VIII comprising heating a solution of telmisartan sodium in dichloromethane at a temperature of about 30° C. to 55° C., preferably heating to reflux, forming a solution; combining n-hexane with the solution forming a mixture; allowing the solution to precipitate; and recovering the precipitate. Preferably, allowing the solution to precipitate comprises allowing the solution to cool to about 20-40° C. Preferably, recovering the precipitate is performed by filtration, more preferably at about room temperature at a pressure below about 100 mmHg by vacuum filtration. Preferably, the precipitate is washed with solvent, more preferably with n-hexane, to obtain wet telmisartan sodium Form VIII.

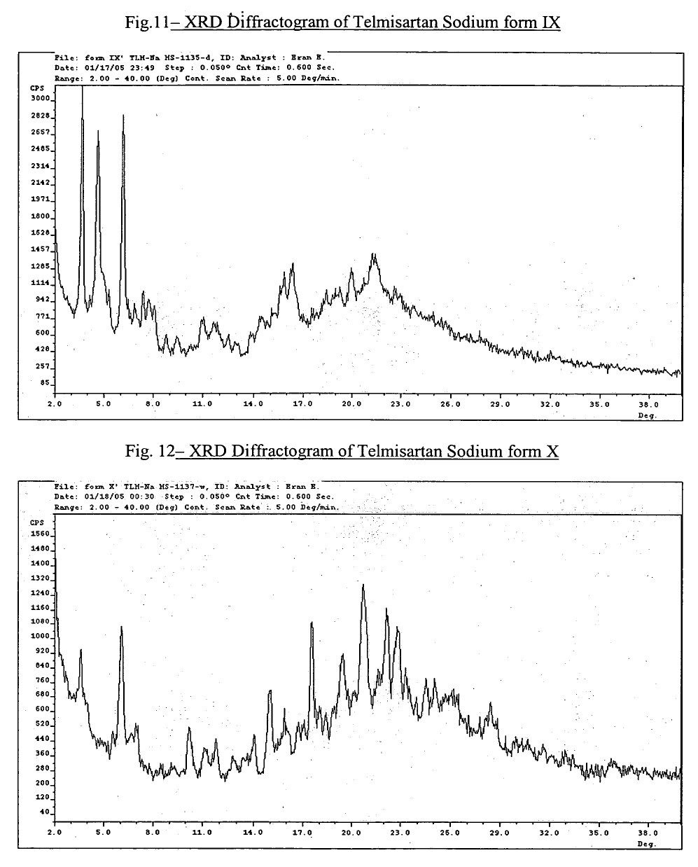

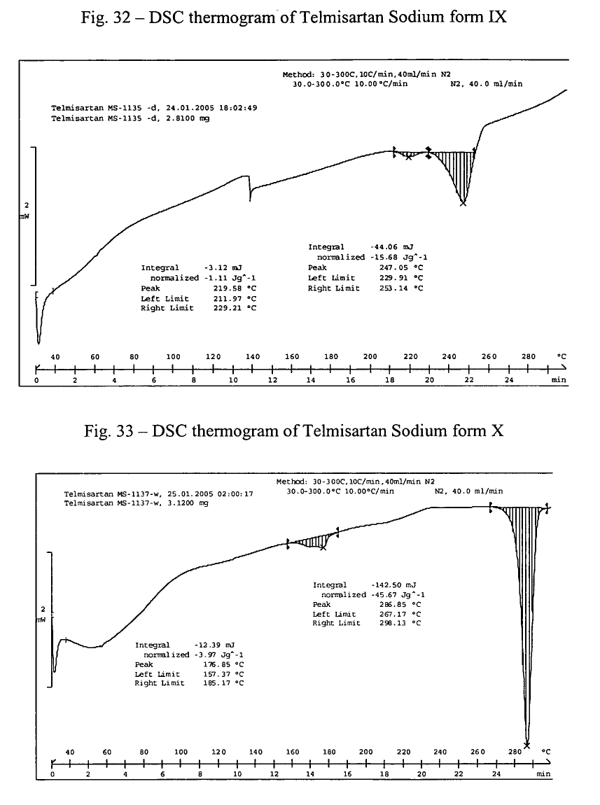

Another embodiment of the invention provides a crystalline form of telmisartan sodium, herein defined as Form IX, which is characterized by an X-ray powder diffraction pattern with peaks at 3.7, 4.6, 6.2, and 16.4 degrees two-theta±0.2 degrees two-theta, substantially as depicted in FIG. 11. Form IX may also be identified by a differential scanning calorimetry thermogram with peaks at about 220 and 247° C., substantially as depicted in FIG. 32.

Another embodiment of the invention provides a process for preparing telmisartan sodium crystal Form IX comprising drying telmisartan sodium crystal Form VIII, preferably at about room temperature at a pressure below about 100 mmHg in a vacuum oven to obtain telmisartan sodium crystal Form IX product.

Another embodiment of the invention provides a crystalline form of telmisartan sodium, herein defined as Form X, which is characterized by an X-ray powder diffraction pattern with peaks at 3.6, 6.1, 15.0, 17.6, 20.7, and 22.1 degrees two-theta±0.2 degrees two-theta, substantially as depicted in FIG. 12. Form X may also be identified by a differential scanning calorimetry thermogram with peaks at about 177 and 287° C., substantially as depicted in FIG. 33.

Another embodiment of the invention provides a process for preparing telmisartan sodium Form X comprising heating telmisartan sodium in dimethylformamide to form a solution, preferably heating to a temperature of from about 75° C. to about 95° C., more preferably to about 90° C.; combining acetonitrile with the solution forming a mixture; allowing the solution to precipitate; and recovering the precipitate. Preferably, allowing the solution to precipitate comprises allowing the solution to cool to about 20-40° C. Preferably, recovering the precipitate is performed by filtration, more preferably at a pressure below about 100 mmHg by vacuum filtration. Preferably, the precipitate is washed with solvent, more preferably with acetonitrile, to obtain wet telmisartan sodium Form X.

Yet another embodiment provides a process for preparing telmisartan sodium Form X comprising heating telmisartan sodium in DMSO to form a solution, preferably heating to a temperature of from about 75° C. to about 95° C., more preferably to about 90° C.; combining acetonitrile with the solution forming a mixture; allowing the solution to precipitate; and recovering the precipitate. Preferably, allowing the solution to precipitate comprises allowing the solution to cool to about 20-40° C.; and adding additional acetonitrile to the cooled mixture. Preferably, recovering the precipitate is performed by filtration, more preferably at a pressure below about 100 mmHg by vacuum filtration. Preferably, the precipitate is washed with solvent, more preferably with acetonitrile, to obtain wet telmisartan sodium Form X.

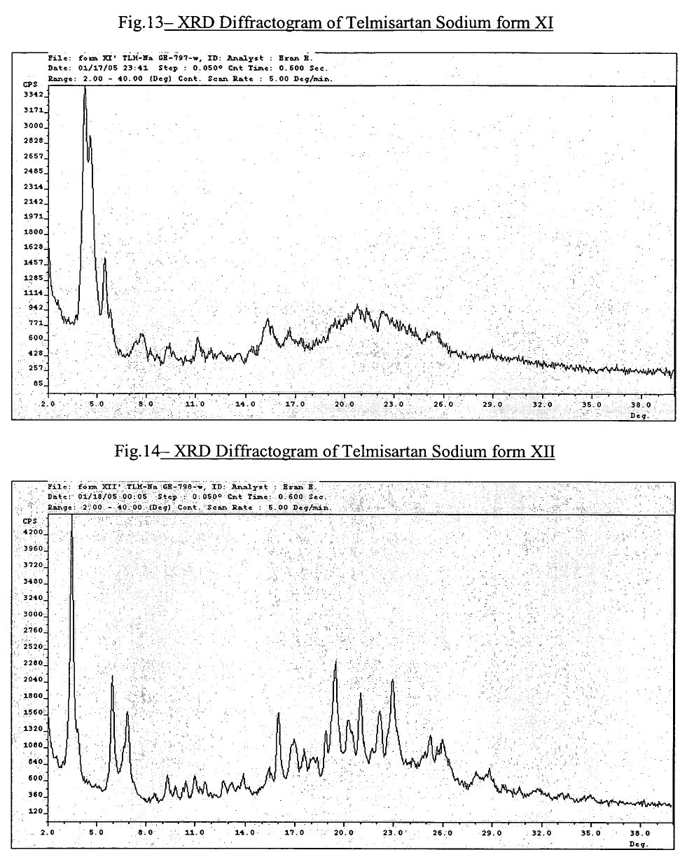

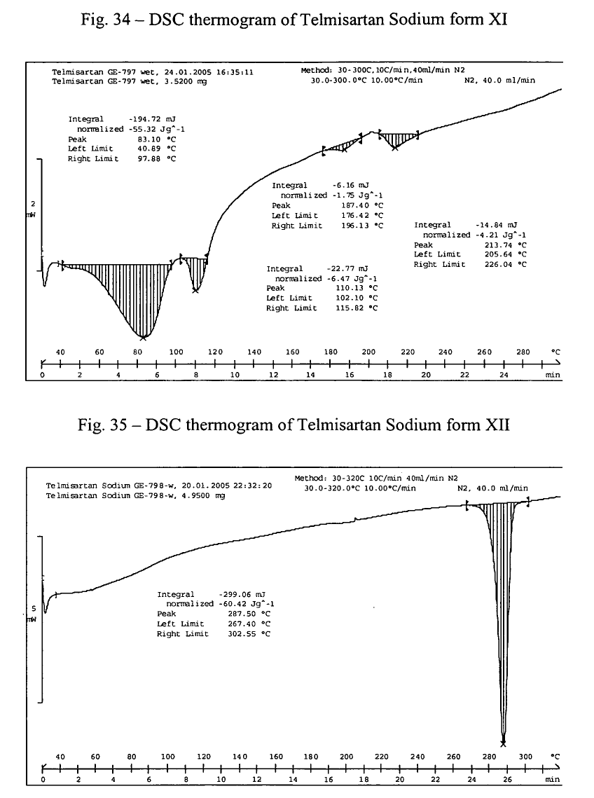

Yet another embodiment of the invention provides a crystalline form of telmisartan sodium, herein defined as Form XI, which is characterized by an X-ray powder diffraction pattern with peaks at 4.2, 4.5, and 5.5 degrees two-theta±0.2 degrees two-theta, substantially as depicted in FIG. 13. Form XI may also be identified by a differential scanning calorimetry thermogram with peaks at about 83, 110, 187, and 214° C., substantially as depicted in FIG. 34.

Another embodiment of the invention provides a process for preparing telmisartan Form XI comprising heating a solution of telmisartan sodium in 1-butanol at a temperature of about 100° C. to 150° C., preferably heating to reflux; combining diethylether with the solution forming a mixture; allowing the mixture to precipitate; and recovering the precipitate. Preferably, allowing the mixture to precipitate comprises allowing the solution to cool, more preferably, cooling to room temperature. Preferably, recovering the precipitate is performed by filtration, more preferably at a pressure below about 100 mmHg by vacuum filtration. Preferably, the precipitate is washed with solvent, more preferably with diethylether, to obtain wet telmisartan sodium crystal Form XI.

Another embodiment of the invention provides a crystalline form of telmisartan sodium, herein defined as Form XII, which is characterized by an X-ray powder diffraction pattern with peaks at 3.5, 6.0, 6.9, 16.1, 19.5, and 23.0 degrees two-theta±0.2 degrees two-theta, substantially as depicted in FIG. 14. Form XII may also be identified by a differential scanning calorimetry thermogram with peaks at about 288° C., substantially as depicted in FIG. 35.

Another embodiment of the invention provides a process for preparing telmisartan sodium Form XII comprising heating a solution of telmisartan sodium in 1-butanol at a temperature of about 100° C. to 150° C., preferably to reflux; combining acetonitrile with the solution forming a mixture; allowing the mixture to precipitate; and recovering the precipitate. Preferably, allowing the solution to precipitate comprises allowing the solution to cool, more preferably, cooling to room temperature. Preferably, recovering the precipitate is performed by filtration, more preferably at a pressure below about 100 mmHg by vacuum filtration. Preferably, the precipitate is washed with solvent, more preferably with acetonitrile, to obtain wet telmisartan sodium crystal Form XII.

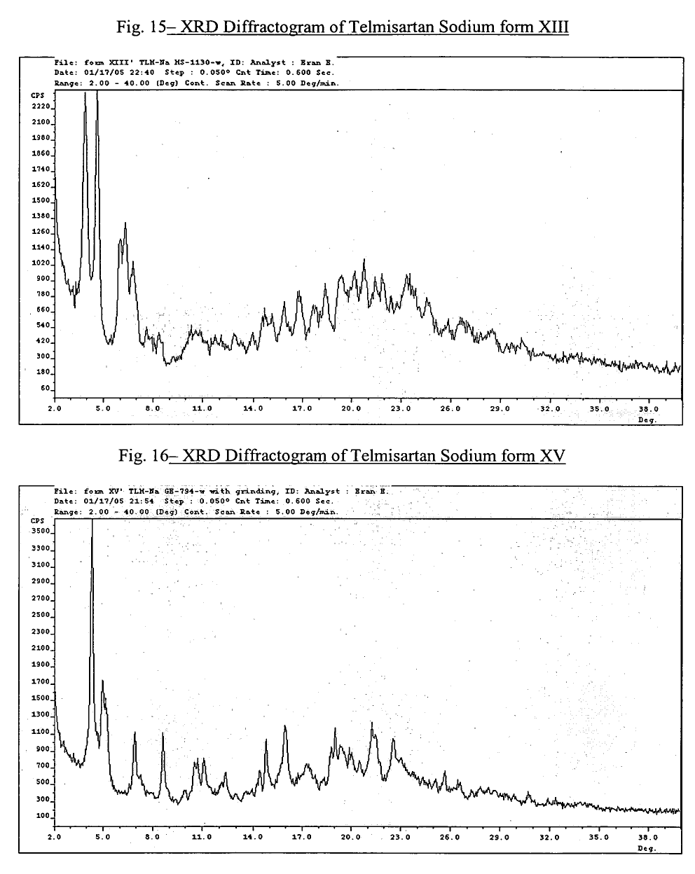

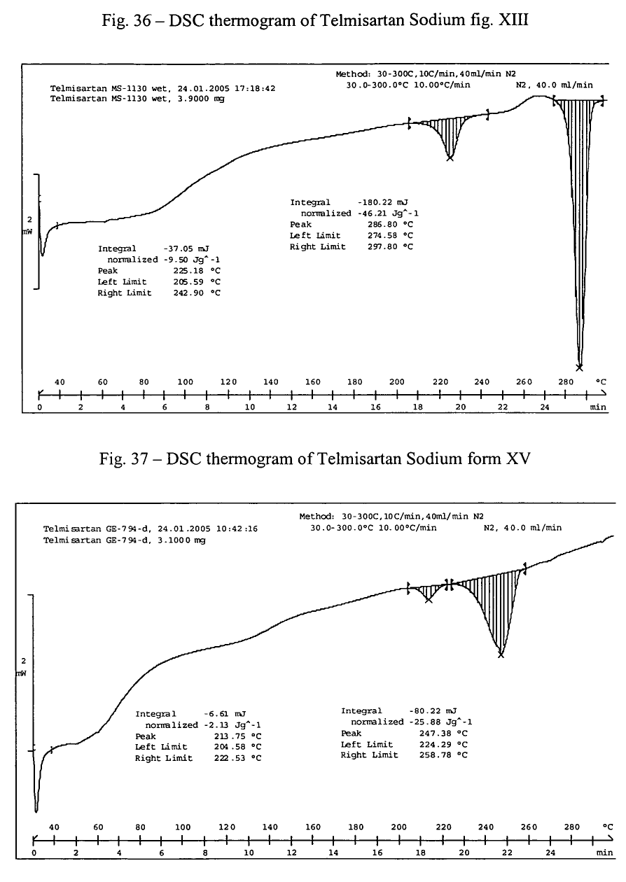

Another embodiment of the invention provides a crystalline form of telmisartan sodium, herein defined as Form XIII, which is characterized by an X-ray powder diffraction pattern with peaks at 3.9, 4.6, 6.0, 6.3, and 6.8 degrees two-theta±0.2 degrees two-theta, substantially as depicted in FIG. 15. Form XIII may also be identified by a differential scanning calorimetry thermogram with peaks at about 225 and 287° C., substantially as depicted in FIG. 36.

Yet another embodiment of the invention provides a process for preparing telmisartan sodium Form XIII comprising heating a solution of telmisartan sodium in dichloromethane at a temperature of about 30° C. to 55° C., preferably to reflux, forming a solution; combining ethylacetate with the solution forming a mixture; allowing the mixture to precipitate; and recovering the precipitate. Preferably, allowing the mixture to precipitate comprises allowing the solution to cool to about 20-40° C. Preferably, recovering the precipitate is performed by filtration, more preferably at about room temperature at a pressure below about 100 mmHg by vacuum filtration. Preferably, the precipitate is washed with solvent, more preferably with ethylacetate, to obtain wet telmisartan sodium crystal Form XIII.

Another embodiment of the invention provides a process for preparing telmisartan sodium Form XIII comprising heating telmisartan sodium in ethanol, preferably heating the solution at a temperature of about 65° C. to 90° C., preferably to reflux; combining acetone with the solution; allowing the solution to precipitate; and recovering the precipitate. Preferably, allowing the solution to precipitate comprises allowing the solution to cool to about 20-40° C., preferably to about room temperature; and adding additional acetone while stirring. Preferably, recovering the precipitate is performed by filtration, more preferably at a pressure below about 100 mmHg by vacuum filtration to obtain wet telmisartan sodium Form XIII.

Another embodiment of the invention provides a process for preparing a mixture of telmisartan sodium Form XIII and telmisartan Form VI comprising drying telmisartan XIII, preferably at about room temperature at a pressure below about 100 mmHg in a vacuum oven to obtain a mixture of telmisartan sodium Form XIII and telmisartan sodium Form VI.

Yet another embodiment of the invention provides a crystalline form of telmisartan sodium, herein defined as Form XV, which is characterized by an X-ray powder diffraction pattern with peaks at 4.3, 5.0, 6.9, 8.6, and 16.0 degrees two-theta±0.2 degrees two-theta, substantially as depicted in FIG. 16. Form XV may also be identified by a differential scanning calorimetry thermogram with peaks at about 214 and 247° C., substantially as depicted in FIG. 37.

Another embodiment of the invention provides a process for preparing telmisartan sodium Form XV comprising heating a solution of telmisartan sodium in methanol at a temperature of about 55° C. to 75° C., preferably to reflux; combining ethylacetate with the solution to form a mixture; allowing the mixture to precipitate; and recovering the precipitate. Preferably, allowing the mixture to precipitate comprises allowing the solution to cool to about 20-40° C. Preferably, recovering the precipitate is performed by filtration, more preferably at a pressure below about 100 mmHg by vacuum filtration. Preferably, the precipitate is washed with solvent, more preferably with ethylacetate to obtain wet telmisartan sodium crystal Form XV. The process may further comprise drying the precipitate.

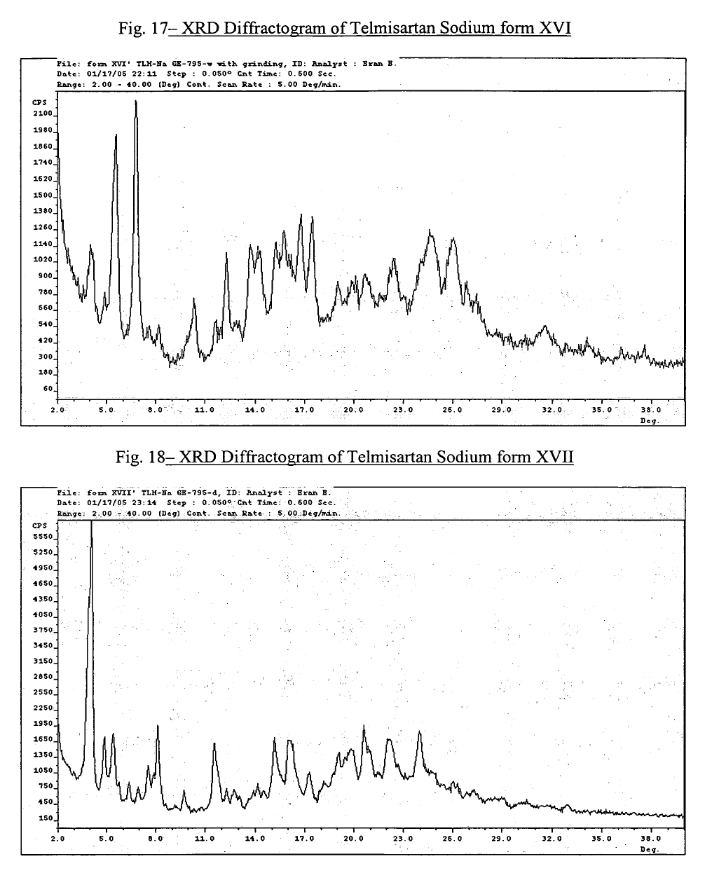

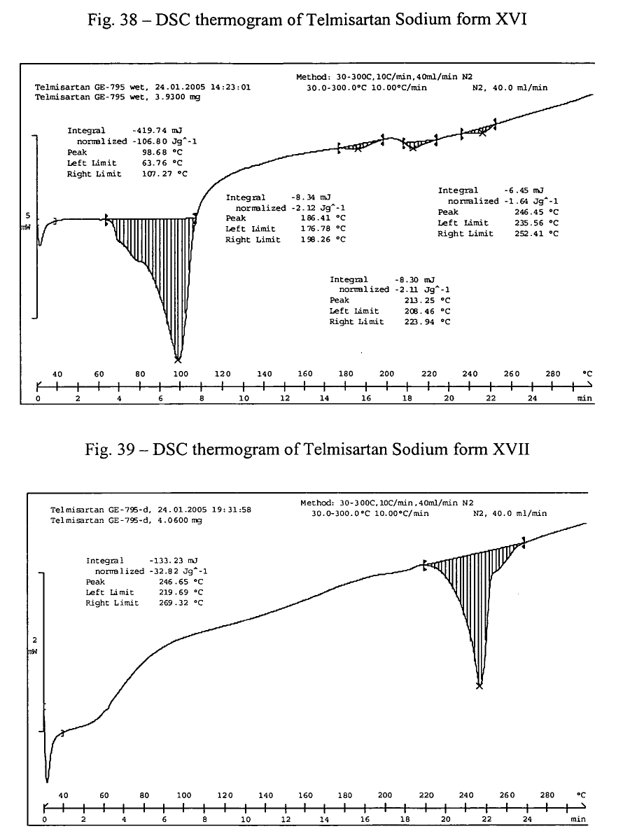

Another embodiment of the invention provides a crystalline form of telmisartan sodium crystal, herein defined as Form XVI, which is characterized by an X-ray powder diffraction pattern with peaks at 4.0, 5.6, 6.8, 10.3, 12.3, 16.8, and 17.5 degrees two-theta±0.2 degrees two-theta, substantially as depicted in FIG. 17. Form XVI may also be identified by a differential scanning calorimetry thermogram with peaks at about 99, 186, 213, and 247° C., substantially as depicted in FIG. 38.

Yet another embodiment of the invention provides a process for preparing telmisartan sodium Form XVI comprising heating a mixture telmisartan sodium Form IV and tetrahydrofuran at a temperature of about 50° C. to 80° C., preferably heating telmisartan sodium Form IV to reflux in tetrahydrofuran, forming a slurry; allowing the slurry to precipitate; and recovering the precipitate. Preferably, allowing the slurry to precipitate comprises allowing the slurry to cool to about 20-40° C. Preferably, recovering the precipitate is performed by filtration, more preferably at a pressure below about 100 mmHg by vacuum filtration. Preferably, the precipitate is washed with solvent, more preferably with tetrahydrofuran, to obtain wet telmisartan sodium crystal Form XVI.

Another embodiment of the invention provides a crystalline form of telmisartan sodium, herein defined as Form XVII, which is characterized by an X-ray powder diffraction pattern with peaks at 4.1, 4.9, 5.3, 8.1, 11.6, 15.2, and 20.7 degrees two-theta±0.2 degrees two-theta, substantially as depicted in FIG. 18. Form XVII may also be identified by a differential scanning calorimetry thermogram with peaks at about 247° C., substantially as depicted in FIG. 39.

Another embodiment of the invention provides a process for preparing telmisartan sodium Form XVII comprising drying telmisartan Form XVI, preferably at about room temperature at a pressure below about 100 mmHg in a vacuum oven to obtain telmisartan sodium Form XVII.

Another embodiment of the invention provides a process for preparing telmisartan sodium Form XVII comprising heating a solution telmisartan sodium and tetrahydrofuran at a temperature of about 50° C. to 80° C., preferably heating to reflux, and recovering the precipitate. Preferably, allowing the solution to precipitate comprises allowing the solution to cool to about 20-40° C. Preferably, recovering the precipitate is performed by filtration, more preferably at a pressure below about 100 mmHg by vacuum filtration. Preferably, the precipitate is washed with solvent, more preferably with tetrahydrofuran, to obtain wet telmisartan sodium crystal Form XVII.

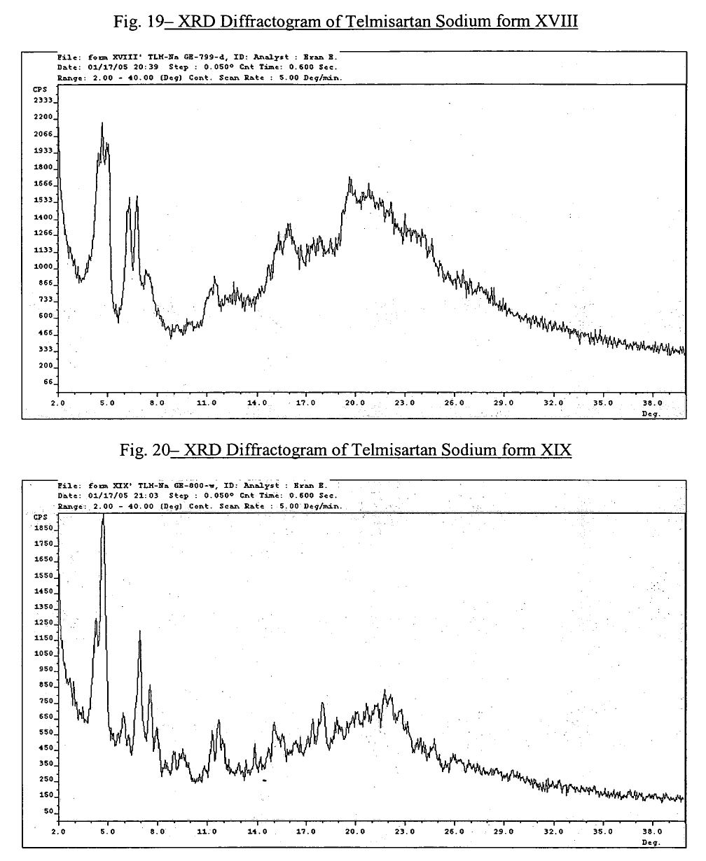

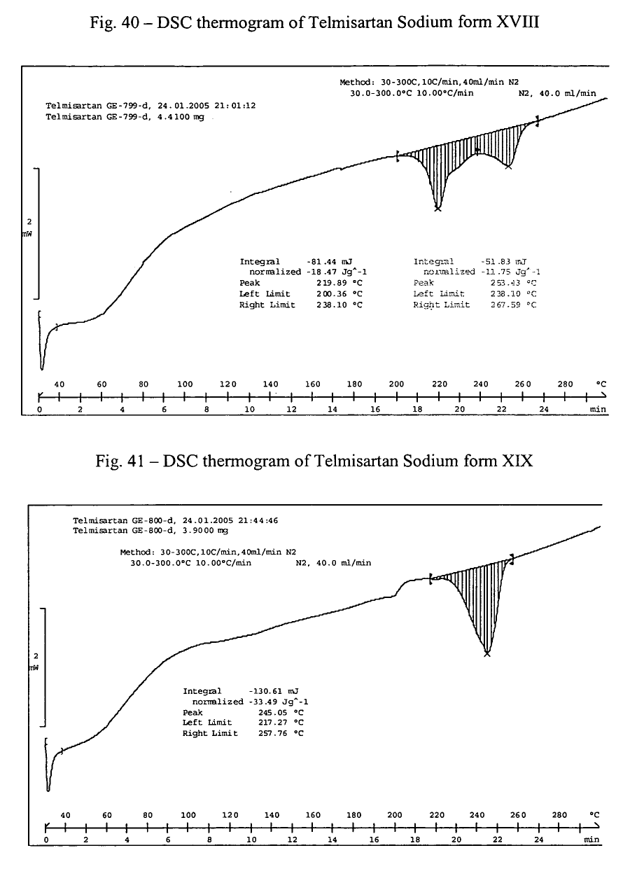

Yet another embodiment of the invention provides a crystalline form of telmisartan sodium, herein defined as Form XVIII, which is characterized by an X-ray powder diffraction pattern with peaks at 4.7, 5.0, 6.3, and 6.8 degrees two-theta±0.2 degrees two-theta, substantially as depicted in FIG. 19. Form XVIII may also be identified by a differential scanning calorimetry thermogram with peaks at about 220 and 253° C., substantially as depicted in FIG. 40.

Another embodiment of the invention provides a process for preparing telmisartan sodium Form XVIII comprising drying telmisartan sodium Form XIII, preferably at about room temperature at a pressure below about 100 mmHg in a vacuum oven forming telmisartan sodium Form XVIII.

One embodiment of the invention provides a crystalline form of telmisartan sodium, herein defined as Form XIX, which is characterized by an X-ray powder diffraction pattern with peaks at 4.3, 4.7, 6.9, and 7.5 degrees two-theta +0.2 degrees two-theta, substantially as depicted in FIG. 20. Form XIX may also be identified by a differential scanning calorimetry thermogram with peaks at about 245° C., substantially as depicted in FIG. 41.

Yet another embodiment of the invention provides a process for preparing telmisartan Form XIX comprising heating telmisartan sodium in dimethylformamide forming a solution, preferably at a temperature of about 75° C. to about 95° C., more preferably at about 90° C.; combining diethylether with the solution forming a mixture; allowing the mixture to precipitate; and recovering the precipitate. Preferably, allowing the mixture to precipitate comprises allowing the solution to cool to about 20-40° C. Preferably, recovering the precipitate is performed by filtration, more preferably at a pressure below about 100 mmHg by vacuum filtration. Preferably, the precipitate is washed with solvent, more preferably with diethylether, to obtain wet telmisartan sodium crystal Form XIX. The process may further comprise drying the precipitate to obtain telmisartan sodium Form XIX product.

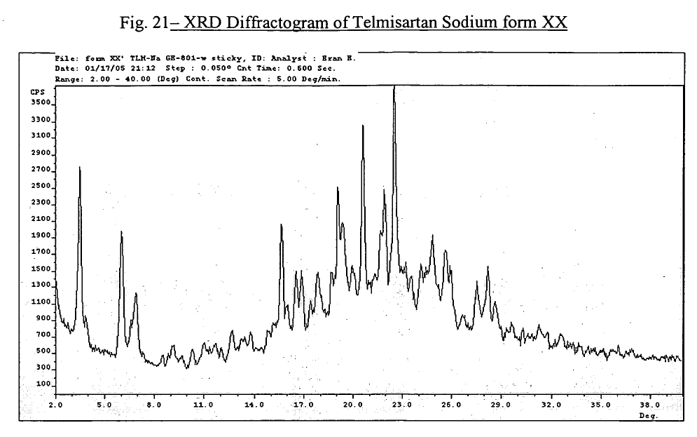

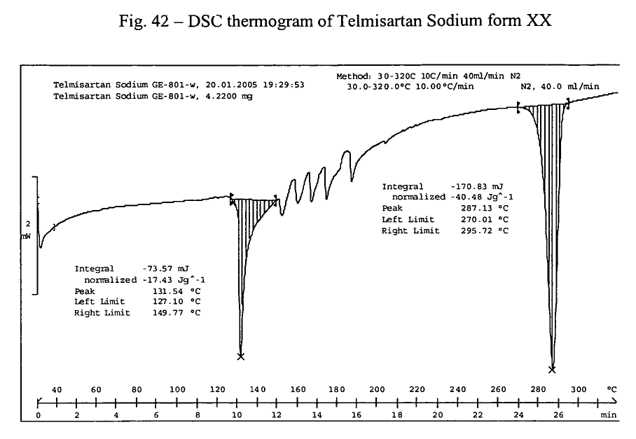

Another embodiment of the invention provides a crystalline form of telmisartan sodium, herein defined as Form XX, which is characterized by an X-ray powder diffraction pattern with peaks at 3.5, 6.0, 6.9, 15.6, 19.0, 20.6, and 22.4 degrees two-theta±0.2 degrees two-theta, substantially as depicted in FIG. 21. Form XX may also be identified by a differential scanning calorimetry thermogram with peaks at about 132 and 287° C., substantially as depicted in FIG. 42.

Another embodiment of the invention provides a process for preparing telmisartan Form XX comprising heating telmisartan sodium in dimethylformamide forming a solution, preferably at a temperature of about 75° C. to about 95° C., more preferably at about 90° C.; combining ethylacetate with the solution forming a mixture; allowing the mixture to precipitate; and recovering the precipitate. Preferably, allowing the mixture to precipitate comprises allowing the solution to cool to about 20-40° C. Preferably, recovering the precipitate is performed by filtration, more preferably at a pressure below about 100 mmHg by vacuum filtration. Preferably, the precipitate is washed with solvent, more preferably with ethylacetate, to obtain wet telmisartan sodium crystal Form XX.

Pharmaceutical formulations of the present invention contain amorphous telmisartan sodium, such as one of those disclosed herein. The invention also provides pharmaceutical compositions comprising crystal forms of telmisartan sodium. Pharmaceutical formulations of the present invention contain the various crystal Forms of telmisartan sodium active ingredients described herein, optionally in admixture with other crystal Form(s) of telmisartan sodium. In addition, to the active ingredient(s), the pharmaceutical formulations of the present invention may contain one or more excipients. Excipients are added to the formulation for a variety of purposes.

Diluents increase the bulk of a solid pharmaceutical composition, and may make a pharmaceutical dosage form containing the composition easier for the patient and caregiver to handle. Diluents for solid compositions include, for example, microcrystalline cellulose (e.g. Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol and talc.

Solid pharmaceutical compositions that are compacted into a dosage form, such as a tablet, may include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate and starch.

The dissolution rate of a compacted solid pharmaceutical composition in the patient's stomach may be increased by the addition of a disintegrant to the composition. Disintegrants include alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®) and starch.

Glidants can be added to improve the flowability of a non-compacted solid composition and to improve the accuracy of dosing. Excipients that may function as glidants include colloidal silicon dioxide, magnesium trisilicate, powdered cellulose, starch, talc and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate.

Flavoring agents and flavor enhancers make the dosage form more palatable to the patient. Common flavoring agents and flavor enhancers for pharmaceutical products that may be included in the composition of the present invention include maltol, vanillin, ethyl vanillin, menthol, citric acid, fumaric acid, ethyl maltol and tartaric acid. Solid and liquid compositions may also be dyed using any pharmaceutically acceptable colorant to improve their appearance and/or facilitate patient identification of the product and unit dosage level.

In liquid pharmaceutical compositions of the present invention, Telmisartan sodium and any other solid excipients are suspended in a liquid carrier such as water, vegetable oil, alcohol, polyethylene glycol, propylene glycol or glycerin.

Liquid pharmaceutical compositions may contain emulsifying agents to disperse uniformly throughout the composition an active ingredient or other excipient that is not soluble in the liquid carrier. Emulsifying agents that may be useful in liquid compositions of the present invention include, for example, gelatin, egg yolk, casein, cholesterol, acacia, tragacanth, chondrus, pectin, methyl cellulose, carbomer, cetostearyl alcohol and cetyl alcohol.

Liquid pharmaceutical compositions of the present invention may also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth and xanthan gum.

Sweetening agents such as sorbitol, saccharin, sodium saccharin, sucrose, aspartame, fructose, mannitol and invert sugar may be added to improve the taste. Preservatives and chelating agents such as alcohol, sodium benzoate, butylated hydroxyl toluene, butylated hydroxyanisole and ethylenediamine tetraacetic acid may be added at levels safe for ingestion to improve storage stability.

According to the present invention, a liquid composition may also contain a buffer such as guconic acid, lactic acid, citric acid or acetic acid, sodium guconate, sodium lactate, sodium citrate or sodium acetate. Selection of excipients and the amounts used may be readily determined by the formulation scientist based upon experience and consideration of standard procedures and reference works in the field.

The solid compositions of the present invention include powders, granulates, aggregates and compacted compositions. The dosages include dosages suitable for oral, buccal, rectal, parenteral (including subcutaneous, intramuscular, and intravenous), inhalant and ophthalmic administration. Although the most suitable administration in any given case will depend on the nature and severity of the condition being treated, the most preferred route of the present invention is oral. The dosages may be conveniently presented in unit dosage form and prepared by any of the methods well-known in the pharmaceutical arts.

Dosage forms include solid dosage forms like tablets, powders, capsules, suppositories, sachets, troches and losenges, as well as liquid syrups, suspensions and elixirs.

The dosage form of the present invention may be a capsule containing the composition, preferably a powdered or granulated solid composition of the invention, within either a hard or soft shell. The shell may be made from gelatin and optionally contain a plasticizer such as glycerin and sorbitol, and an opacifying agent or colorant.

The active ingredient and excipients may be formulated into compositions and dosage forms according to methods known in the art.

A composition for tableting or capsule filling may be prepared by wet granulation. In wet granulation, some or all of the active ingredients and excipients in powder form are blended and then further mixed in the presence of a liquid, typically water, that causes the powders to clump into granules. The granulate is screened and/or milled, dried and then screened and/or milled to the desired particle size. The granulate may then be tableted/compressed, or other excipients may be added prior to tableting, such as a glidant and/or a lubricant.

A tableting composition may be prepared conventionally by dry blending. For example, the blended composition of the actives and excipients may be compacted into a slug or a sheet and then comminuted into compacted granules. The compacted granules may subsequently be compressed into a tablet.

As an alternative to dry granulation, a blended composition may be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate and colloidal silica. The proper use of these and other excipients in direct compression tableting is known to those in the art with experience and skill in particular formulation challenges of direct compression tableting.

A capsule filling of the present invention may comprise any of the aforementioned blends and granulates that were described with reference to tableting, however, they are not subjected to a final tableting step.

Moreover, amorphous telmisartan sodium is preferably formulated for administration to a mammal, preferably a human, via injection. Amorphous telmisartan sodium may be formulated, for example, as a viscous liquid solution or suspension, preferably a clear solution, for injection. The formulation may contain solvents. Among considerations for such solvent include the solvent's physical and chemical stability at various pH levels, viscosity (which would allow for syringeability), fluidity, boiling point, miscibility and purity. Suitable solvents include alcohol USP, benzyl alcohol NF, benzyl benzoate USP and Castor oil USP. Additional substances may be added to the formulation such as buffers, solubilizers, antioxidants, among others. Ansel et al., Pharmaceutical Dosage Forms and Drug Delivery Systems, 7^{th} Ed.

The present invention also provides pharmaceutical compositions of amorphous form of telmisartan sodium and polymorph Forms of telmisartan sodium to be used in methods of treatment of a mammal in need thereof. Telmisartan is a non-peptide angiotensin II receptor (type AT1) antagonist approved for the treatment of hypertension. A pharmaceutical composition as in the present invention comprising one or more forms of telmisartan sodium selected from the group consisting of amorphous telmisartan sodium and crystalline telmisartan sodium forms 0, I to XIII, XV to XX, may be used in a method of treatment of a mammal comprising administering to a mammal suffering from hypertension a therapeutically effective amount of such pharmaceutical composition.

Having described the invention with reference to certain preferred embodiments, other embodiments will become apparent to one skilled in the art from consideration of the specification. The invention is further defined by reference to the following examples describing in detail the preparation of the compounds of the present invention. It will be apparent to those skilled in the art that many modifications, both to materials and methods, may be practiced without departing from the scope of the invention.

### EXAMPLES

Instruments:

X-Ray Diffraction

X-Ray diffraction (XRD) analysis was performed on X-Ray powder diffractometer, Scintag, equipped with a variable goniometer, Cu-tube, solid state detector and a round standard sample holder with round zero background. The scanning parameters were from a range of about 2-40 degrees two θ(±0.2 degrees) and a continuous scan at a rate of about 3 degrees/minute. One of ordinary skill in the art understands that experimental differences may arise due to differences in instrumentation, sample preparation, or other factors.

Thermal Analysis

Differential scanning calorimetry (DSC) was conducted using a Mettler Toledo DSC821e with a sample weight of about 3-5 mg, a heating rate of about 10° C./min., and using a 3 holed crucible.

### Example 1

A 250ml round bottom flask was loaded with TLM-Me (8 gr), abs. EtOH (80 ml) and NaOH solution (28%, 1. leq.). The mixture was heated to reflux for 10.5 hrs then it was divided to two portions:

A) water (40 ml) was added and the EtOH was evaporated. The aqueous solution was lyophilized to obtain amorphous TLM-Na (3.46 gr).

B) Abs. EtOH (25 ml) was added and the solvents were evaporated to obtain amorphous TLM-Na (3.25 gr).

### Example 2

A 250 ml round bottom flask was loaded with telmisartan (5 g), ethanol (96%, 100 ml) and sodium hydroxide (47%, 0.8 ml). The solution was stirred at room temperature for 0.5 hour and the solvents were evaporated to obtain amorphous telmisartan sodium (5.5 g).

### Example 3

A 1 L round bottom flask was loaded with telmisartan (20 g), ethanol (96%, 200 ml) and sodium hydroxide (47%, 2.2 ml). The mixture was stirred at room temperature for 3.5 hours, and then filtered to abtain a solution. The mother liqueur was evaporated to obtain amorphous telmisartan sodium (20.41 g).

### Example 4

Telmisartan sodium (Form IV, 3 g) was dissolved in absolute ethanol (15 ml) by heating to reflux. Hexane (75 ml) was combined over a period of 25 minutes, the mixture was cooled to room temperature and stirred for 7 hours. A precipitate product formed and was isolated by vacuum filtration and washed with hexane (10 ml) to obtain telmisartan sodium Form 1 (2.26 g). The wet sample was dried in a vacuum oven at 50° C. overnight to obtain telmisartan sodium Form 1 (1.28 g, 43% yield).

### Example 5

A 250 ml round bottom flask was loaded with telmisartan sodium (Form IV, 3 g) and absolute ethanol (15 ml) and the mixture was heated to reflux. Ethylacetate (75 ml) was combined over a period of 30 minutes, the solution was cooled to room temperature, and stirred overnight. Ethylacetate was combined (100 ml) and the mixture was stirred for a few hours.

The product was isolated by vacuum filtration to obtain wet telmisartan sodium Form 0 (1.87 g). The sample was dried in a vacuum oven at 50° C. for 20 hours to obtain telmisartan sodium Form 0 (1.44 g, 48% yield).

### Example 6

Telmisartan sodium (6 g) was dissolved in isopropyl alcohol (20 ml) by heating to reflux and the solution was cooled to room temperature. Methyl tertiary butyl ether (MTBE) was then added (20 ml). A sticky precipitation was obtained. The sticky precipitate was heated again to reflux and isopropyl alcohol (20 ml) was combined. After 1 hour a homogeneous slurry was obtained, and was cooled to room temperature. The product was isolated by vacuum filtration, washed with MTBE (10 ml) and dried in vacuum oven at 50° C. overnight to obtain telmisartan sodium Form 1 (3 g, ˜55% yield).

### Example 7

Telmisartan sodium (amorphous, 4 g) was dissolved in isopropyl alcohol (30 ml) by heating the mixture to reflux, turbid solution was obtained. Methyl tertiary butyl ether (MTBE) (100 ml) was combined and the mixture was cooled to room temperature. The product was isolated by vacuum filtration, washed with MTBE (7 ml) and dried in a vacuum oven at 50° C. overnight to obtain a mixture of telmisartan sodium Form I and Form II (3.04 g, ˜76% yield).

### Example 8

Telmisartan sodium (amorphous, 4 g) was dissolved in absolute ethanol (10 ml) by heating to reflux. Methyl tertiary butyl ether (MTBE) (50 ml) was combined over a period of 1.33 hours and the mixture was cooled to room temperature. The product was isolated by vacuum filtration, washed with MTBE (10 ml) and dried in vacuum oven at 50° C. overnight to obtain telmisartan sodium Form 11 (3.04 g, ˜83% yield).

### Example 9

Telmisartan sodium (Form IV, 3g) was dissolved in absolute ethanol (15 ml) by heating to reflux. Diethyl ether (75 ml) was combined over a period of 1.25 hours, the mixture was cooled to room temperature and stirred for 6 hours. The product was isolated by vacuum filtration, washed with diethyl ether (10 ml) to obtain a mixture of wet telmisartan sodium Form I and Form 11 (4.32 g). The sample was then dried in a vacuum oven at 50° C. overnight to obtain a mixture of telmisartan sodium Form I and Form 11 (2.34 g, ˜78% yield).

### Example 10

Telmisartan sodium (amorphous, 4 g) was,dissolved in methanol (10 ml) by heating to reflux. Methyl tertiary butyl ether (50 ml) was combined during 1.5 hours, the mixture was cooled to room temperature and stirred overnight. The product was isolated by vacuum filtration and dried in vacuum oven at 50° C. overnight to obtain telmisartan sodium Form III (1.9 g, ˜45% yield).

### Example 11

Wet telmisartan sodium Form XI (9.93 g) was dried in a vacuum oven at 50° C. for 20 hours to obtain telmisartan sodium Form IV (2.77 g, 92% yield).

### Example 12

A two liter round bottom flask was loaded with telmisartan (80 g), absolute methanol (800 ml) and sodium hydroxide (47%, 8.83 ml). The solution was stirred at room temperature for 2 hours, filtered and evaporated. The residue (88.68 g) was dissolved in absolute ethanol (200 ml) by heating to reflux. Methyl tertiary butyl ether (MTBE) (1 L) was combined over a period of 3.5 hours and the mixture was cooled to room temperature and stirred for about 1 hour. The product was isolated by vacuum filtration, washed with MTBE (40 ml) and dried in a vacuum oven at 50° C. for 16.5 hours to obtain telmisartan sodium Form IV (79.45 g, 95% yield).

### Example 13

A 250 ml round bottom flask was loaded with telmisartan sodium (Form IV, 3 g) and toluene (100 ml) and heating the slurry to reflux. The slurry was stirred for 3 hours then cooled to room temperature and stirred over night. The product was isolated by vacuum filtration, washed with toluene (10 ml) and dried in vacuum oven at 50° C. for 21 hours to obtain telmisartan sodium Form V (2.62 g, 87% yield).

### Example 14

A 250 ml round bottom flask was loaded with amorphous telmisartan sodium (3 g) and toluene (60 ml) and heating the mixture to reflux forming a slurry. The slurry was stirred for 1 hour then cooled to room temperature and stirred over night. The product was isolated by vacuum filtration, washed with toluene, (5 ml) and dried in a vacuum oven at 50° C. for 21 hours to obtain Telmisartan sodium Form V (2.46 g, 82% yield).

### Example 15

A 250 ml round bottom flask was loaded with telmisartan sodium (Form IV, 3 g) and toluene (60 ml) heating the mixture to reflux forming a slurry. The slurry was stirred for 1.5 hours then cooled to room temperature and stirred over night. The product was isolated by vacuum filtration, washed with toluene, (5 ml) and dried in vacuum oven at 50° C. for 23 hours to obtain telmisartan sodium Form V (2.86 g, 95% yield).

### Example 16

A 250 ml round bottom flask was loaded with telmisartan sodium Form IV (3 g) and methyl ethyl ketone (100 ml) heating the mixture to reflux forming a slurry. The slurry was stirred for 2.5 hours then cooled to room temperature and stirred over night. The product was isolated by vacuum filtration, washed with methyl ethyl ketone (10 ml) and dried in vacuum oven at 50° C. for 21 hours to obtain telmisartan sodium Form VI (2.72 g, 91% yield).

### Example 17

Telmisartan sodium (Form IV, 3 g) was dissolved in absolute ethanol (15 ml) by heating to reflux. Acetonitrile (75 ml) was combined during 50 minutes. After another 10 minutes the mixture was cooled to room temperature and stirred for 6 hours. The product was isolated by vacuum filtration, washed with acetonitrile (3.5 ml) and dried in vacuum oven at 50° C. for 13 hours to obtain telmisartan sodium Form VI (1.06 g, 35% yield).

### Example 18

Telmisartan sodium Form XII (4.22 g) was dried in a vacuum oven at 50° C. for 20 hours to obtain telmisartan sodium Form VI (2.1 g, 70% yield).

### Example 19

Telmisartan sodium Form XX (2.97 g) was dried in a vacuum oven at 50° C. overnight to obtain telmisartan sodium Form VI (1.85 g, 62% yield).

### Example 20

Wet telmisartan sodium form X (4.33 g) was dried in a vacuum oven at 50° C. for 16 hours to obtain telmisartan sodium Form VI (2.37 g, 80% yield).

### Example 21

Wet telmisartan sodium Form X (2.29 g) was dried in vacuum oven at 50° C. for 17 hours to obtain telmisartan sodium Form VI (1.62 g, 63% yield).

### Example 22

A 250 ml round bottom flask was loaded with telmisartan sodium (Form IV, 3 g) and dimethylsulfoxide (15 ml) and the mixture was heated to 75° C. Ethylacetate (92 ml) was combined over a period of 35 minutes and the solution was cooled to room temperature. Ethylacetate (150 ml) was combined and the mixture was stirred over night. The product was isolated by vacuum filtration, washed with ethylacetate (6 ml) to obtain wet telmisartan sodium Form VII, which dried in vacuum oven at 50° C. for 24 hours to obtain telmisartan sodium Form VII (98.7% yield).

### Example 23

A 250 ml round bottom flask was loaded with telmisartan sodium (Form IV, 3 g) and dichloromethane (40 ml) and the mixture was heated to reflux. Methyl tertiary butyl ether (MTBE) (92 ml) was combined during 35 minutes and the mixture was cooled to room temperature and stirred over night. The product was isolated by vacuum filtration, washed with MTBE (4 ml) to obtain wet telmisartan sodium Form VIII, which dried in vacuum oven at 50° C. for 24 hours to obtain telmisartan sodium Form VIII (98.8% yield).

### Example 24

A 250 ml round bottom flask was loaded with telmisartan sodium (Form IV, 3 g) and dichloromethane (40 ml) and the mixture was heated to reflux. n-Hexane (100 ml) was combined over a period of 35 minutes and the mixture was cooled to room temperature and stirred overnight. The product was isolated by vacuum filtration, washed with n-Hexane (4 ml) to obtain wet telmisartan sodium Form VIII (3.77 g).

### Example 25

Telmisartan sodium Form VIII (3.21 g) was dried in a vacuum oven at 50° C. for 24 hours to obtain telmisartan sodium Form IX (2.57 g, 100% yield).

### Example 26

A 250 ml round bottom flask was loaded with telmisartan sodium (Form IV, 3 g) and dimethylformamide (15 ml) and the mixture was heated to 90° C. for dissolution. Acetonitrile (75 ml) was combined during 10 minutes. The temperature was reduced to 68° C. and the mixture was cooled to room temperature and stirred 2 hours. The product was isolated by vacuum filtration, washed with actonitrile (10 ml) to obtain wet telmisartan sodium form X (4.94 g).

### Example 27

A 250 ml round bottom flask was loaded with telmisartan sodium (Form IV, 3 g) and dimethylsulfoxide (15 ml) and the mixture was heated to 90° C. for dissolution. Acetonitrile (75 ml) was combined during 20 minutes and the mixture was cooled to room temperature. Additional acetonitrile (50 ml) was combined and the mixture was stirred for 2 hours. The product was isolated by vacuum filtration, washed with acetonitrile (2 ml) to obtain wet telmisartan sodium Form X (2.68 gr).

### Example 28

A 250 ml round bottom flask was loaded with telmisartan sodium Form IV (3 g) and 1-butanol (15 ml) and the mixture was heated to reflux for dissolution. Diethylether (75 ml) was combined and the temperature was reduced to 41° C. and the mixture was cooled to room temperature and stirred for 2 hours. The product was isolated by vacuum filtration, washed with diethylether (10 ml) to obtain wet telmisartan sodium Form XI.

### Example 29

A 250 ml round bottom flask was loaded with telmisartan sodium (amorphous, 3 g) and 1-butanol (15 ml) and the mixture was heated to reflux for dissolution. Acetonitrile (75 ml) was combined, the temperature reduced to 70° C. and the mixture was cooled to room temperature and stirred for 2 hours. The product was isolated by vacuum filtration, washed with acetonitrile (10 ml) to obtain wet telmisartan sodium Form XII (4.83 g).

### Example 30

A 250 ml round bottom flask was loaded with telmisartan sodium (Form IV, 3 g) and dichloromethane (40 ml) and the mixture was heated to reflux for dissolution. Ethylacetate (150 ml) was combined over a period of 15 minutes, the temperature reduced to 55° C., the mixture was cooled to room temperature, and stirred for 19 hours. The product was isolated by vacuum filtration, washed with ethylacetate (10 ml) to obtain wet telmisartan sodium Form XIII (9.12 g).

### Example 31

A 250 ml round bottom flask was loaded with telmisartan sodium (Form IV, 3 g) and absolute ethanol (15 ml) and the mixture was heated to reflux. Acetone (75 ml) was combined over a period of 50 minutes. The temperature reduced to 58° C. and the solution was cooled to room temperature, and stirred over night. Acetone was combined (100 ml) and the mixture was stirred for another night. The product was isolated by vacuum filtration to obtain wet telmisartan sodium Form XIII (2.06 g).

### Example 32

Telmisartan sodium Form XIII (1.92 g) was dried in a vacuum oven at 50° C. for 24 hours to obtain telmisartan sodium mixture of Form XIII and Form VI (1.13 g, 40% yield).

### Example 33

A 250 ml round bottom flask was loaded with telmisartan sodium (Form IV, 3 g) and methanol (12 ml) and the mixture was heated to reflux for dissolution. Ethylacetate (70 ml) was combined over a 10 minute period, the solution was cooled to room temperature without forming a precipitate. Ethylacetate (200 ml) was combined and the mixture was stirred for 24 hours.

The product was isolated by vacuum filtration, washed with ethylacetate (10 ml) to obtain wet telmisartan sodium Form XV which dried in vacuum oven at 50° C. for 24 hrs to obtain telmisartan sodium Form XV (1.7 g, 57% yield).

### Example 34

A 250 ml round bottom flask was loaded with telmisartan sodium Form IV (3 g) and tetrahydrofuran (45 ml) and heated to reflux forming a slurry. The slurry was stirred for 1 hour then cooled to room temperature and stirred for 2 hours. The product was isolated by vacuum filtration, washed with tetrahydrofuran (10 ml) to obtain wet telmisartan sodium Form XVI.

### Example 35

Wet telmisartan sodium Form XVI (5.39 g) was dried in a vacuum oven at 50° C. for 22 hours to obtain telmisartan sodium Form XVII (2.44 g, 81% yield).

### Example 36

Telmisartan sodium Form XIII (8.74 g) was dried in a vacuum oven at 50° C. for 24 hours to obtain telmisartan sodium Form XVIII (2.82 g, 94% yield).

### Example 37

A 250 ml round bottom flask was loaded with telmisartan sodium (Form IV, 3 g) and dimethylformamide (15 ml) and the mixture was heated to 90° C. for dissolution. Diethyl ether (75 ml) was combined during 5 minutes. The temperature reduced to 41° C. and the mixture was cooled after 0.5 hour to room temperature, and stirred for 3 hours.

The product was isolated by vacuum filtration, washed with diethylether (10 ml) to obtain wet telmisartan sodium Form XIX which is dried in a vacuum oven at 50° C. for 24 hours to obtain telmisartan sodium Form XIX (1.9 g, 63% yield).

### Example 38

A 250 ml round bottom flask was loaded with telmisartan sodium (Form IV, 3 g) and dimethylformamide (15 ml) and the mixture was heated to 90° C. for dissolution. Ethylacetate (75 ml) was combined over a 20 minute period. The temperature was reduced to 68° C., the mixture was cooled to room temperature, and stirred over night. The product was isolated by vacuum filtration, washed with ethylacetate (10 ml) to obtain wet telmisartan sodium Form XX (3.5 g).

### Example 39

Telmisartan sodium (amorphous, 3 g) was dissolved in tetrahydrofuran (20 ml) by heating to reflux. After 10 minutes precipitation occurred and the mixture was stirred at reflux for another 0.5 hr. The mixture was cooled to room temperature and stirred 3 days. The product was isolated by vacuum filtration, washed with tetrahydrofuran (6 ml) and dried in vacuum oven at 50° C. for 21 hrs to obtain Telmisartan sodium crystalline form XVII (35.5% yield).

1. An amorphous form of telmisartan sodium.

2. A crystalline form of telmisartan sodium characterized by having peaks at X-ray powder diffraction patterns selected from the group consisting of:
2.4, 4.7, 6.2, 7.1, 7.4, 15.3, and 22.3 degrees 20, ±0.2 degrees 2θ.
4.1, 5.0, 6.2, 7.0, and 15.2 degrees 20, ±0.2 degrees 2θ;
4.6, 7.4, 7.7, 15.3, and 22.4 degrees 20, ±0.2 degrees 2θ;
4.0, 4.3, and 5.1 degrees 20, ±0.2 degrees 2θ;
4.5, 4.9, 7.3, 15.2, 19.3, and 22.4 degrees 20, ±0.2 degrees 2θ;
4.1, 5.2, 5.6, 7.6, 8.0, 8.4, 11.9, 15.9, 19.7, and 23.2 degrees 20, ±0.2 degrees 2θ;
6.0, 6.8, 10.4, 11.8, 16.7, 17.7, 19.3, 20.8, and 23.5 degrees 20, ±0.2 degrees 2θ;
5.6, 10.0, 14.8, 17.4, and 20.4 degrees 20, ±0.2 degrees 2θ;
4.0, 4.4, 6.3, and 6.8 degrees 20, ±0.2 degrees 2θ;
3.7, 4.6, 6.2, and 16.4 degrees 20, ±0.2 degrees 2θ;
3.6, 6.1, 15.0, 17.6, 20.7, and 22.1 degrees 20, ±0.2 degrees 2θ;
4.2, 4.5, and 5.5 degrees 20, ±0.2 degrees 2θ;
3.5, 6.0, 6.9, 16.1, 19.5, and 23.0 degrees 20, ±0.2 degrees 2θ;
3.9, 4.6, 6.0, 6.3, and 6.8 degrees 20, ±0.2 degrees 2θ;
4.3, 5.0, 6.9, 8.6, and 16.0 degrees 20, ±0.2 degrees 2θ;
4.0, 5.6, 6.8, 10.3, 12.3, 16.8, and 17.5 degrees 20, ±0.2 degrees 2θ;
4.1, 4.9, 5.3, 8.1, 11.6, 15.2, and 20.7 degrees 20, ±0.2 degrees 2θ;
4.7, 5.0, 6.3, and 6.8 degrees 20, ±0.2 degrees 2θ;
4.3, 4.7, 6.9, and 7.5 degrees 20, ±0.2 degrees 2θ; and
3.5, 6.0, 6.9, 15.6, 19.0, 20.6, and 22.4 degrees 20, ±0.2 degrees 2θ;

3. A pharmaceutical composition of telmisartan sodium prepared using one or more forms of the telmisartan sodium forms of any one of claims 1 and 2 and at least one excipient.

4. A method of treatment of a mammal, comprising administering to a mammal suffering from hypertension a therapeutically effective amount of the pharmaceutical composition of claim 3.

5. Telmisartan sodium according to any one of claims 1 and 2 for use in therapy.

6. Telmisartan sodium according to any one of claims 1 and 2 for use in treating hypertension.

7. Use of telmisartan sodium according to any one of claims 1 and 2 for use in manufacture of a medicament for treating hypertension.
